(19) United States
(12) Patent Application Publication
(10) Pub. No.: US 2025/0269087 A1
(43) Pub. Date: Aug. 28, 2025

(54) BIOLOGICAL TISSUE AND METHOD OF MANUFACTURING

(71) Applicant: BioCreations Medical, LLC, San Antonio, TX (US)

(72) Inventors: Oliver BURCKHARDT, Philadelphia, PA (US); Daniel PINTO, San Antonio, TX (US); Laura Bordallo CASTILLO, San Antonio, TX (US); Andrey AGASHCHUK, San Antonio, TX (US)

(21) Appl. No.: 19/208,449

(22) Filed: May 14, 2025

Related U.S. Application Data
(63) Continuation of application No. PCT/US2023/079706, filed on Nov. 14, 2023.
(60) Provisional application No. 63/383,574, filed on Nov. 14, 2022.
(60) Provisional application No. 63/383,578, filed on Nov. 14, 2022.

(51) Int. Cl. A61L 27/38; A61J 3/00; A61L 27/24

(52) U.S. Cl. CPC A61L 27/3804; A61J 3/00; A61L 27/24

(57) ABSTRACT

A processed adipose tissue is described, having an extracellular matrix derived from human adipose tissue and adipocyte cells having an intact adipocyte composition of at least 10%. A method of processing adipose tissue and a media loading tool are also described.

### CROSS-REFERENCE TO RELATED APPLICATIONS

This application is a National Stage of International Patent Application No. PCT/US2023/079706, filed Nov. 14, 2023, which claims priority to U.S. provisional application No. 63/383,574 filed on Nov. 14, 2022 and U.S. provisional application No. 63/383,578 filed on Nov. 14, 2022, the disclosures of which are hereby incorporated by reference in their entireties.

### BACKGROUND OF THE INVENTION

The field of aesthetic or reconstructive surgical procedures are often the result of disease, trauma, or injuries that damage the local soft tissue. These defects can lead to a loss of normal tissue function, emotional trauma, and pain (Coleman, S. R. 2016. Plastic and Reconstructive Surgery 118, 108S-120S). Alternatively, cosmetic applications include small-volume (facial contouring) or large-volume (breast and buttocks) treatments that use either synthetic or natural fillers. Current clinical procedures often use autologous fat transfers or commercially available fillers to aid in the repair (Coleman, S. R. 2016. Plastic and Reconstructive Surgery 118, 108S-120S., Gir, P. et al. 2012. Plastic and Reconstructive Surgery 130, 249-258.) Autologous fat transfers use the patient's own adipose tissue, via liposuction, to fill voids while minimizing host immune rejection and promoting adipogenesis (Serra-Renom, J. M. et al. 2010. Plastic and Reconstructive Surgery 125, 12-18.) While advantageous, using a heterogeneous unprocessed fat can lead to donor site morbidity from liposuction (Patrick Jr, C. W. 2001. The Anatomical Record 263, 361-366.), poor survival of the remaining adipocytes, which can lead to necrosis (Young, D. A. et al. 2012. Biomed Mater 7, 024104.), and delivery of free lipids and tissue pieces can result in cyst formation and calcifications (Kokai, L. E. et al. 2020. Plast Reconstr Surg Glob Open 8, e2574.). These complications could lead to poor aesthetic results such as contour irregularities, tissue bunching, inflammation or even more severe problems such as fat embolisms, stroke, and/or blindness, depending on the location of the injections (Vasavada, A. et al. 2023. Autologous Fat Grafting For Facial Rejuvenation., StatPearls Publishing LLC.)

Commercially available fillers have grown in popularity due to the increased demand for cosmetic surgical procedures, with recent research providing insight into current advancements in this field (Choi, J. H. et al. 2010. Tissue Eng Part B Rev 16, 413-426.) Synthetic and natural materials have been extensively used as soft tissue fillers with limited success. Synthetic polymers, such as polyethylene glycol or polylactic acid, offer injectable and/or compositional control and are biologically inert, leading to reduced host integration, granuloma formation (Cohen, G. et al. 2009. Journal of drugs in dermatology: JDD 8, 486-489.), and/or immune rejection (Levenberg, S. et al. 2004. Curr Top Dev Biol 61, 113-134., Cronin, K. J. et al. 2004. Plastic and Reconstructive Surgery 113, 260-269., Patrick, C. W. et al. 1999. Tissue Eng 5, 139-151.)

Despite this, synthetic polymers lack appropriate native tissue composition and mechanical properties. Further, biodegradable synthetic polymers exhibit poor mechanical properties (Young, D. A. et al. 2012. Biomed Mater 7, 024104.), poor volume retention (Patrick Jr, C. W. 2001. The Anatomical Record 263, 361-366.) and the biodegradable compounds can accumulate resulting in immune responses or even toxicity (Fu, K. et al. 2000. Pharmaceutical Research 17, 100-106., Mäder, K. et al. 1996. Biomaterials 17, 457-461.)

To better recapitulate native tissue, natural fillers containing extracellular matrix (ECM) have shown increased volume retention over time, host integration, reduced immune responses, and adipogenesis for in vitro (Flynn, L. E. 2010. Biomaterials 31, 4715-4724.), in vivo (Young, D. A. et al. 2012. Biomed Mater 7, 024104., Han, T. T. Y. et al., 2015, Biomaterials 72, 125-137.), and clinical studies (Gold, M. H. et al. 2020. J Cosmet Dermatol 19, 1044-1056., Dige A. et al. 2019. Gastroenterology 156, 2208-2216 e2201.) Native ECM retains the structural and biological properties that enhance cell-cell and cell-matrix interactions for increased cell growth and tissue regeneration (Boudreau, N. et al. 2006. Cell 125, 429-431.) These outcomes have led to the increased interest of minimally manipulated adipose tissues that retain the native ECM, for use as adipose tissue allografts (Banyard, D. A. et al. 2019. Regenerative Medicine and Plastic Surgery: Skin and Soft Tissue, Bone, Cartilage, Muscle, Tendon and Nerves, 71-89, Springer International Publishing.).

However, the use of chemical, biological and physical processing in the manufacture of ECM-retaining tissue can lead to the disruption of ECM proteins, structure or potentially damage the sample. There is a need in the art for a minimally manipulated injectable processed tissue that retains the native adipocyte structure and ECM. Embodiments described herein meet this need.

### SUMMARY OF THE INVENTION

In one embodiment, a processed adipose tissue has an extracellular matrix derived from human adipose tissue, and adipocyte cells having an intact adipocyte composition of at least 10%. In one embodiment, the intact adipocyte composition of at least 30%. In one embodiment, the intact adipocyte composition is between 30% and 70%. In one embodiment, the intact adipocyte composition is between 30% and 55%. In one embodiment, the intact adipocyte composition is between 45% and 70%. In one embodiment, the tissue structural proteins include at least one of collagen, glycoproteins, and proteoglycans retained from the human adipose tissue. In one embodiment, the tissue includes decellularized tissue having a reduced amount of allogeneic components, cellular components and free lipids. In one embodiment, the tissue is configured for implantation or injection into a patient. In one embodiment, the tissue is configured for passing through an opening between 1 mm and 4.5 mm. In one embodiment, a method of processing adipose tissue includes the steps of freezing and thawing a human adipose tissue, separating the human adipose tissue into pieces by mechanical grinding, serial rinsing the human adipose tissue with a non-ionic surfactant, a crystalloid solution and sterile water, and dispensing the human adipose tissue into packaging using a loading tool. In one embodiment, the loading tool includes a jar having a proximal end opening, a distal end opening and a chamber disposed therebetween, a piston actuated by a lever and configured to slide distally and flush along a wall of the chamber, and a stand configured to stabilize the piston below the lever and form hinged connection with the lever. In one embodiment, the proximal and distal end openings are each surrounded at least partially by a threaded surface. In one embodiment, the loading tool includes an adapter is configured to connect to at least one of the proximal and distal openings of the jar, the adapter having a first sealable port. In one embodiment, the piston has a second sealable port. In one embodiment, the method includes debriding the human adipose tissue to remove extraneous or bruised tissue. In one embodiment, the method includes rinsing the human adipose tissue with a non-ionic surfactant, sterile crystalloid solution, and sterile water by dispensing the human adipose tissue into centrifuge tubes and centrifuging the human adipose tissue. In one embodiment, the method includes rinsing the human adipose tissue with a non-ionic surfactant, sterile crystalloid solution, and sterile water by dispensing the human adipose tissue into a sealable container and gently shaking the container. In one embodiment, the method includes rinsing the tissue in a non-ionic surfactant at least two times. In one embodiment, the method includes rinsing the tissue in a crystalloid solution at least two times. In one embodiment, the method includes rinsing the tissue in sterile water at least two times. In one embodiment, the method includes drying the human adipose tissue in a centrifuge tube comprising a drying sieve. In one embodiment, the method includes decanting the tissue after each rinse and removing any decanted liquid and cellular components. In one embodiment, the method includes homogeneously dispensing the human adipose tissue in containers using the loading tool. In one embodiment, the method includes packing the containers in a sterile pouch.

In one embodiment, a media loading tool includes a jar having a proximal end opening, a distal end opening and a chamber disposed therebetween; a piston actuated by a lever and configured to slide distally and flush along a wall of the chamber; and a stand configured to stabilize the piston below the lever and form a hinged connection with the lever. In one embodiment, the proximal and distal end openings are each surrounded at least partially by a threaded surface. In one embodiment, an adapter is configured to connect to at least one of the proximal and distal openings, the adapter having a first sealable port. In one embodiment, the piston has a second sealable port.

### BRIEF DESCRIPTION OF THE DRAWINGS

The foregoing purposes and features, as well as other purposes and features, will become apparent with reference to the description and accompanying figures below, which are included to provide an understanding of the invention according to various embodiment and constitute a part of the specification, in which like numerals represent like elements, and in which:

FIG. 1A depicts a flow diagram illustrating embodiments of a method for producing the adipose tissue filler allograft according to one embodiment, FIG. 1B depicts a flow diagram illustrating a method for producing the adipose tissue filler allograft according to one embodiment, and FIG. 1C depicts a flow diagram illustrating a method of processing an adipose tissue according to one embodiment.

FIGS. 2A and 2B depict alternate side views of a loading tool assembly according to one embodiment.

FIG. 2C is a cross-sectional view of the loading tool jar according to one embodiment.

FIGS. 2D and 2E depict alternate views of a first adapter according to one embodiment, and FIG. 2F depicts alternate views of a second adapter according to one embodiment.

FIG. 3A depicts a visual inspection chart for the color inspection according to one embodiment.

FIG. 3B depicts a visual inspection chart for the excess free lipid inspection according to one embodiment.

FIG. 4 depicts a graph showing the DNA quantification results according to one embodiment.

FIG. 5 depicts a graph showing the average DNA content in all samples tested according to one embodiment.

FIG. 6 depicts a table showing the average component quantification in percentages according to one embodiment.

FIG. 7A and FIG. 7B depict the component amounts of water, lipid, and solid (including ECM) in grams according to one embodiment.

FIG. 8A depicts a graph showing relative amounts of ECM protein expression between an embodiment as compared to native adipose tissue. FIG. 8B depicts a heat map showing the major ECM components expressed in an embodiment as compared to native adipose tissue.

FIG. 9A-9D depict four histological images from adipose tissue. FIG. 9A and FIG. 9B depict staining from unprocessed tissue. FIG. 9C and FIG. 9D depict staining according to one embodiment.

FIG. 10A-10D depict four histological images from adipose tissue. FIG. 10A and FIG. 10B depict staining from unprocessed tissue. FIG. 10C and FIG. 10D depict staining from an embodiment.

FIGS. 11-14 depict four histological images from adipose tissue. FIG. 11 and FIG. 12 depict staining from unprocessed tissue. FIG. 13 and FIG. 14 depict staining from an embodiment.

FIGS. 15-17 depict images of histological staining of samples of the invention at different time points. FIG. 15A shows a staining at 2 months. FIG. 15B shows a staining at 3 months. FIG. 16 shows a staining at greater than 4 months. FIG. 17 shows a staining at 6 months.

FIG. 18 depicts images of histological staining of samples of the invention at different time points. FIG. 18A shows a staining at greater than 2 months. FIG. 18B shows a staining at greater than 3 months. FIG. 18C shows a staining 4.5 months.

FIG. 19 depicts images of histological staining of samples of the invention at different time points. FIG. 19A shows a staining at less than 1 month. FIG. 19B shows a staining at 1.5 months. FIG. 19C shows a staining at 2.5 months.

FIG. 20 depicts a graph showing the mechanical hardness over time of various samples of the invention.

FIG. 21 shows the mechanical hardness over time of one sample of the invention from one donor.

FIG. 22 shows the mechanical hardness over rime of one sample of the invention from a second donor.

FIG. 23 depicts images of staining from a sample of human ASCs grown in control media in the absence of a test sample at A) 24 hrs., B) 48 hrs., C) 1 week, D) 2 weeks, and (E) Oil Red O stained at 2 weeks.

FIG. 24 depicts images of staining of human ASCs grown in differentiation media in the absence of a test sample at A) 24 hrs., B) 48 hrs., C) 1 week, D) 2 weeks, and E) Oil Red O stained at 2 weeks.

FIG. 25 depicts images of staining of human ASCs grown in control media in the presence of a test sample at A) 24 hrs., B) 48 hrs., C) 1 week, D) 2 weeks, and E) Oil Red O stained at 2 weeks.

FIG. 26 depicts images of staining of humans ASCs in control media in the presence of a test sample at A) 24 hrs., B) 48 hrs., C) 1 week, D) 2 weeks, and E) Oil Red O stained at 2 weeks.

FIG. 27 depicts images of staining of humans ASCs in control media in the presence of a test sample at A) 24 hrs., B) 48 hrs., C) 1 week, D) 2 weeks, and E) Oil Red O stained at 2 weeks.

FIG. 28 depicts images of staining of humans ASCs grown in differentiation media in the presence of a test sample at A) 24 hrs., B) 1 week, C) 2 weeks, D) Oil Red O stained at 2 weeks.

FIG. 29 depicts images of staining of humans ASCs grown in differentiation media in the presence of a test sample at A) 24 hrs., B) 1 week, C), D) 2 weeks, and E), and F) Oil Red O stained at 2 weeks.

FIG. 30 depicts images of staining of humans ASCs grown in differentiation media in the presence of a test sample at A) 24 hrs., B) 1 week, C), D) 2 weeks, E), and F) Oil Red O stained at 2 weeks.

FIG. 31 depicts images of staining of humans ASCs grown in control media in the presence of a test sample identified as “Conventional” at A) 24 hrs., B) 48 hrs., C), 1 week, D) 2 weeks, and E) Oil Red O stained at 2 weeks.

FIG. 32 depicts images of staining of humans ASCs grown in differentiation media in the presence of a test sample identified as “Conventional” at A) 24 hrs., B) 1 week, C), D) 2 weeks, and E), and F) Oil Red O stained at 2 weeks.

FIG. 33 depicts a table presenting the results of the human ASC induced differentiation assay of various samples of the invention and conventional samples.

FIG. 34 depicts a graph showing the absorbance values for samples of a test embodiment of the invention, conventional samples and control samples.

FIG. 35 depicts images of test wells with the test sample identified as “Conventional” at A) before test sample was added, B) 24hrs after test sample was added, C) 48 hrs., D) 72 hrs. and E) with 1% EV stain.

FIG. 36 depicts images of test wells with the test sample identified as “invention” at A) before test sample was added, B) 24hrs after test sample was added, C) 48 hrs., D) 72 hrs. and E) with 1% EV stain.

FIG. 37 depicts images of control wells with primary Human Dermal Blood Endothelial Cells (HDBEC) and complete growth medium identified as “Positive Control” at A) before test sample was added, B) 24 hrs after test sample was added, C) 48 hrs., and D) 72 hrs.

FIG. 38 depicts images of control wells with primary HDBEC and basal media identified as “Negative Control” at A) before test sample was added, B) 24 hrs after teste sample was added, C) 48 hrs., and D) 72 hrs.

FIG. 39 depicts a table presenting the results from the endothelial proliferation assay.

FIG. 40 depicts a graph presenting the results from the endothelial proliferation assay.

FIG. 41 depicts a table presenting the results from the endothelial proliferation assay averaged across samples of the invention, samples from a conventional tissue and the positive and negative controls.

FIG. 42 depicts a graph presenting the results from the endothelial proliferation assay averaged across samples of the invention, samples from a conventional tissue and the positive and negative controls.

FIG. 43 shows image J analysis using adiposoft plugin to calculate adipocyte area.

FIG. 44 shows equation 1.

FIG. 45 shows an image with five regions that were analyzed for each sample stain for a comprehensive assessment.

FIG. 46 shows a first processed tissue H&E Stain.

FIG. 47 shows a second processed tissue H&E Stain.

FIG. 48 shows a conventional processed tissue H&E Stain.

FIG. 49 is a table of the Adipocyte Composition range for the first embodiment and the Adipocyte Composition range for the second embodiment.

FIG. 50 is a graph showing compositional analysis of adipose tissue shows lipid content (75-80%), water content (15-20%), and protein content (3-5%).

FIG. 51 is a table of first allograft embodiment compositional analysis.

FIG. 52 is a graph showing first tissue component quantification for representative Donor 1.

FIG. 53 is a table of second allograft embodiment compositional analysis.

FIG. 54 is a table of adipose tissue compositional comparison excluding data for 1 representative donor 3.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a minimally manipulated tissue filler allograft.

One strategy to retain native extracellular matrix (ECM) in a minimally manipulated adipose tissue is decellularization. Decellularization is a process that aims to improve biocompatibility or minimize an immune response by removing tissue resident cells while leaving the ECM intact (Fu, R. H. et al. 2014. Cell Transplant 23, 621-630.) The process steps can vary, but generally requires serial washes in chemical and biological agents that can range from a few days to more than a week (Flynn, L. E. 2010. Biomaterials 31, 4715-4724., Aamodt, J. M. et al. 2016 Biomaterials 86, 68-82., Hinderer S. et al. 2016. Adv Drug Deliv Rev 97, 260-269.) The use of a chemical, biological, and physical processing can lead to the disruption of ECM proteins (e.g. glycosaminoglycans) (Reing J. E. et al. 2010. Biomaterials 31, 8626-8633.), structure (Sesli, M. et al. 2018. Turk J Biol 42, 537-547.), or potentially damaging the sample (Hayes, D. J. et al. 2022. Biomaterials and Biosystems 7, 100053.) In general, decellularization of adipose tissue requires precise control over a series of processing steps (approximately 2-5 days depending on the tissue and protocol) (Wang, L. et al. 2013. Acta Biomaterialia 9, 8921-8931., Mohiuddin, O. A. et al. 2019. 57-70 Springer International Publishing) to ensure minimal damage to the residual ECM while ensuring all the cellular components are removed (Choi, J. S. et al. 2011. Journal of Biomedical Materials Research Part A 97A, 292-299.) The use of minimally manipulated adipose tissues requires less processing steps while still providing native adipose extracellular matrix (ECM) and tissue integrity for soft tissue fillers. Minimal manipulation of adipose tissue, or processing that does not alter the original relevant characteristics relating to the tissue's utility for reconstruction, repair, or replacement (21 CFR 1271.3(f)(1)) offers a safe (Services, U. S. D. o. H. a. H. 2020 ed Food and Drug Administration), and a standardized approach providing clinicians with an off-the shelf, minimally manipulated adipose tissue product (MMAP).

The present invention relates to a minimally manipulated adipose tissue allograft able to be injected or surgically implanted into patients requiring soft tissue supplementation. The adipose tissue allograft herein retains the native ECM and adipocytes of the donor tissue. Further, the adipose tissue allograft also retains structural proteins such as collagen, glycoproteins and proteoglycans. The retained cellular components are necessary to maintain the structural integrity and volume of the allograft. The adipose tissue allograft also contains a highly reduced amount of cellular and allogenic components, such as DNA and nucleic acids, that can induce immune responses which, if severe enough, can lead to immune rejection of the allograft. The allograft further retains components such as ECM, lipids and water, the amounts of which can be varied through the embodiments of the manufacturing process described herein to produce various embodiments of the invention. The adipose tissue filler allograft indeed demonstrates higher levels of adipocyte cells in histological staining, as well as ECM structure and structural proteins, as well as a highly reduced amount of DNA and nucleic acids (See example 2).

The present disclosure further provides embodiments of a method of preparing a minimally manipulated adipose tissue allograft that can retain at least a portion of the intracellular components. The method herein comprises several steps, discussed in detail below, to lyse the tissue of cellular and allogenic components while avoiding the degeneration and wash-out of structural proteins. Cellular and allogenic components are removed through a freeze-thaw process, mechanical grinding process, and mild detergent, sterile saline and sterile water rinses. These processes help to weaken and/or destroy the cell membrane of the cells in the tissue and hence decellularize the tissue. Further, the rinses also separate the intracellular components from the rest of the tissue. These processes are also gentle enough to aid the retention of the necessary structural components of the tissue, for example extracellular matrix, adipocytes and structural proteins. The methods described herein are minimally manipulative methods that are within 361 HCT/P guidelines.

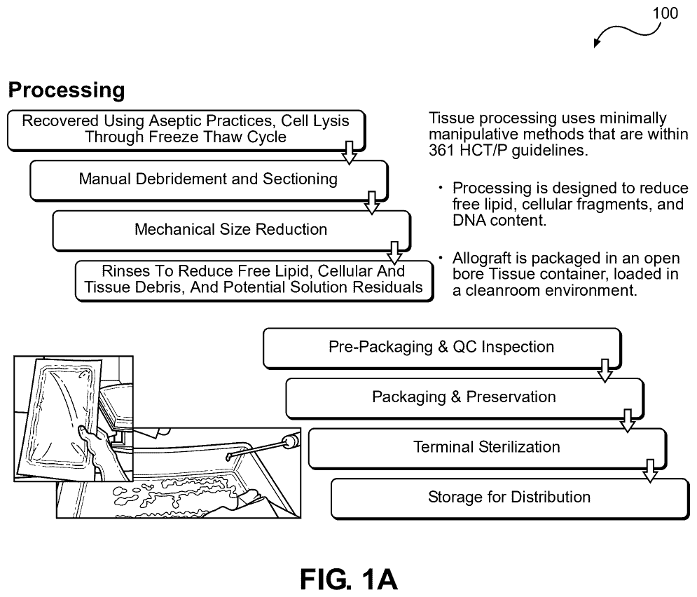

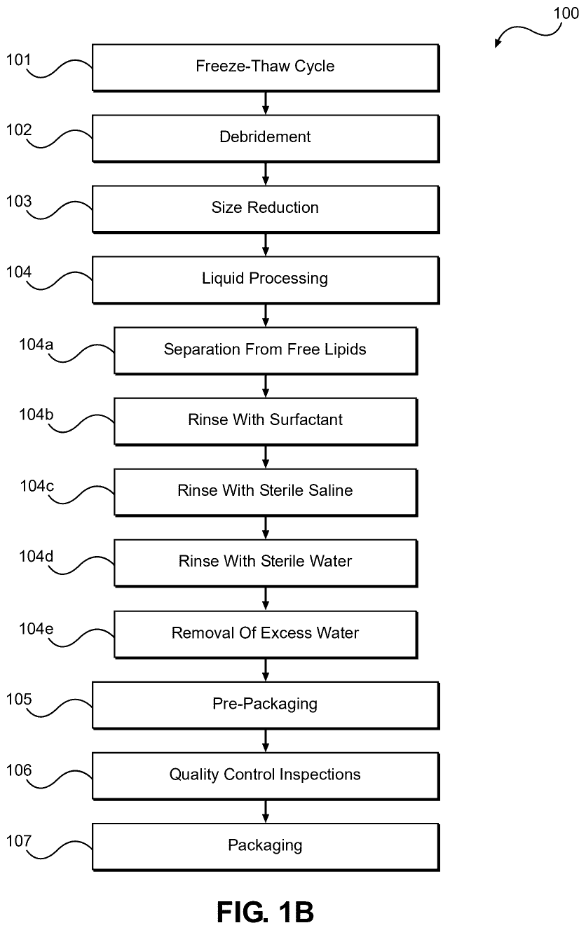

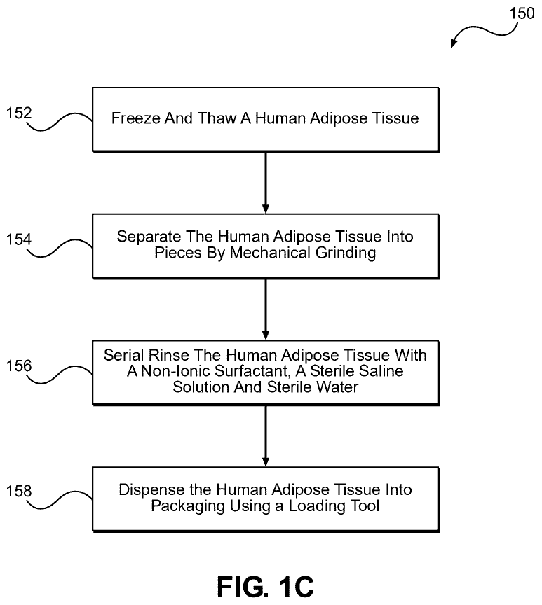

Referring now to FIG. 1A, a method for processing adipose tissue 100 generally includes the steps of a freeze-thaw cycle (step 101) human donor adipose tissue debridement and sectioning (step 102), size reduction (step 103), liquid processing (step 104), pre-packaging preservation (step 105), quality control inspections (step 106), and packaging (step 107). These processes combine in a unique way to influence the quantities of the components of the tissue including adipocytes, free lipid, extracellular matrix, water and oxygen, as well as remove voids and homogenize the allograft.

The method begins by harvesting human cadaveric adipose tissue from a donor using any suitable method. Suitable methods may include, but are not limited to, aspiration, dissection, scraping and other surgical methods known in the art. In other embodiments donor tissue can be harvested and stored at freezing temperatures.

Referring now to FIG. 1B, in one embodiment, step 101 comprises freezing the harvested donor tissue and allowing the harvested donor tissue to thaw for a period of time not exceeding 120 hours. This freeze-thaw cycle is the first step in decellularizing the tissue, as cell membranes may be broken down during the freeze-thaw.

Step 102 comprises debriding the tissue to remove any extraneous or bruised tissue. The tissue is debrided onto a sterile field, with the amount of time that the tissue is open being consistently recorded. The adipose tissue is measured, weighed and inspected for bruises and extraneous tissues, like visible fascia, arteries, veins or muscle. The tissue is debrided using methods known in the art to remove all extraneous tissues and bruised adipose tissue. Suitable methods may include, but are not limited to, the use of scalpels, forceps, scrapers and other methods known in the art. Any extraneous and bruised tissue may be discarded.

Step 103 comprises mechanically separating the tissue into small pieces. In some embodiments, separation can be performed by cutting, grinding, crushing or any other suitable method. In some embodiments, the donor tissue can be cut into pieces. These pieces can be cubes of approximately 4 cm by 4 cm or smaller. Further separation can be performed by feeding the adipose tissue cubes into a mechanical grinder. In some embodiments, the mechanical grinder can be a meat grinder or any other suitable grinder known in the art. Any excess connective tissue obtained from the tissue separation process may be discarded.

Mechanical grinding contributes to the decellularization process of the cell, the shearing force of the grinder having the ability to tear the cell membranes of some cells. Mechanical grinding of the adipose tissue also allows the product to be subjected to enough force such that the tissue is separated into fine particles. This provides the necessary injectability to the product allowing it to pass through containers, syringes or cannulas of 1.5 mm-4.5 mm diameters or greater. In one embodiment, the processed tissue is injectable though a 1.5 mm diameter opening. In one embodiment, the processed tissue is injectable though a 2 mm diameter opening. In one embodiment, the processed tissue is injectable though a 2.5 mm diameter opening. In one embodiment, the processed tissue is injectable though a 3 mm diameter opening. In one embodiment, the processed tissue is injectable though a 3.5 mm diameter opening. In one embodiment, the processed tissue is injectable though a 4 mm diameter opening. In one embodiment, the processed tissue is injectable though a 4.5 mm diameter opening. Further, the mechanical grinding step also subjects the tissue to a gentle enough force that allows for keeping at least 10% of adipocyte cells intact in the tissue. Intact adipocyte cells in one embodiment maintain native adipocyte structure. Intact adipocyte cells in one embodiment maintain native adipocyte functionality for cushioning and support. Intact adipocyte cells in one embodiment maintain tissue structure and extracellular matrix (ECM) of the adipocyte cells. Intact adipocyte cells in one embodiment maintain tissue structure, ECM, triglycerides, and connective tissue of adipocyte cells. In one embodiment, tissue is processed with adipocyte cells having an intact adipocyte composition of at least 10%. In one embodiment, tissue is processed with adipocyte cells having an intact adipocyte composition of at least 15%. In one embodiment, tissue is processed with adipocyte cells having an intact adipocyte composition of at least 20%. In one embodiment, tissue is processed with adipocyte cells having an intact adipocyte composition of at least 25%. In one embodiment, tissue is processed with adipocyte cells having an intact adipocyte composition of at least 30%. In one embodiment, tissue is processed with adipocyte cells having an intact adipocyte composition of at least 40%. In one embodiment, tissue is processed with adipocyte cells having an intact adipocyte composition of at least 50%. In one embodiment, tissue is processed with adipocyte cells having an intact adipocyte composition of at least 55%. In one embodiment, tissue is processed with adipocyte cells having an intact adipocyte composition of at least 60%. In one embodiment, tissue is processed with adipocyte cells having an intact adipocyte composition of at least 65%. In one embodiment, tissue is processed with adipocyte cells having an intact adipocyte composition of at least 70%. The adipocytes help to maintain the structural integrity and volume of the allograft, making it a better implant for soft tissues. Other methods known in the art for size reduction, for example cryomilling, do not allow for the retention of adipocyte cells.

Step 104 comprises the liquid processing of the product. This step is broken down into several sub-steps which include serial rinses with a non-ionic surfactant, sterile crystalloid solution, and sterile water. After each rinse, the tissue is decanted to separate it from unwanted liquids or cellular components.

Step 104a comprises separating the tissue from free lipids. In some embodiments, the separation can be performed by dispensing the ground adipose tissue in equal parts into centrifuge tubes. The tissue can then be centrifuged for durations at a speed of 900-3655 RCF. Decanting the adipose tissue through a sieve or other methods known in the art can separate the free lipids from the adipose tissue. Any decanted liquid, oil or cell pellets is discarded. The tissue can then be rinsed with sterile warm water. The tissue is dispensed in equal parts back into the centrifuge tubes and sterile warm water is added at approximately a 1:1 ratio of sterile warm water to tissue. The centrifuge tubes can be shaken gently to disperse the adipose tissue into the water. The tubes can then be centrifuged at ambient temperature at 900-3655 RCF. The adipose tissue can then be decanted through a sieve and any decanted liquid, oil or cell pellet is discarded.

Step 104b comprises rinsing the adipose tissue with a non-ionic surfactant. This step further contributes to cell lysing, the surfactant aiding in weakening the cell membranes to decellularize the tissue. In some embodiments, the surfactant product used can be a 0.01% Triton X-100 solution. In some embodiments, the rinse is performed by dispensing the adipose tissue into centrifuge tubes and adding in the 0.01% Triton X-100 solution. The solution can be added in a 1:1 ratio of solution to adipose tissue. The centrifuge tubes can be shaken gently for a time, such as 100, 200, 300, 400, 500, 600, 800 or 1,000 seconds or more. The centrifuge tubes can then be centrifuged at speeds of 900-3655 RCF. In some embodiments the rinse can be performed by dispensing the adipose tissue in a sealable container. The 0.01% Triton X-100 solution is then added in an approximately 1:1 ratio of solution to adipose tissue. The sealable container is then shaken a few times during a rinse time (e.g. 1, 2, 3, 4, 5, 6, 8, 10 minutes or more). The adipose tissue can then be decanted through a sieve and any decanted liquid, oil or cell pellet is discarded. The surfactant rinse can be repeated as needed.

The adipose tissue is dried. In some embodiments, drying can be performed by centrifuging the tissue. Centrifuging may be performed for e.g. 1, 2, 3, 4, 5, 6, 8 or 10 minutes at a speed of 900-3655 RCF. The adipose tissue is decanted through a sieve and any decanted liquid, oil, or cell pellet is discarded.

Step 104c comprises rinsing the tissue in a sterile crystalloid solution. The adipose tissue is rinsed with a sterile crystalloid solution. In some embodiments, sterile crystalloid solution can be a 0.9% sterile saline solution. In some embodiments, the rinse may be performed by dispensing the adipose tissue into centrifuge tubes. A 0.9% sterile saline solution is added to the tubes in an approximately 1:1 ratio of solution to tissue. The centrifuge tubes are shaken gently. The centrifuge tubes are then centrifuged at ambient temperature at a speed of 900-3655 RCF. In some embodiments, the rinse may be performed by dispensing the adipose tissue in a sealable container. A 0.9% sterile saline solution is added to the adipose tissue in an approximately 1:1 ratio of solution to tissue. The sealable container is shaken gently a few times during rinse time. After rinsing, the tissue may then be decanted through a sieve, and any decanted liquid, oil or cell pellet is discarded. The sterile saline rinse may be repeated multiple times.

Step 104d comprises rinsing the tissue in sterile water. The adipose tissue is rinsed in sterile water. In some embodiments, the sterile water may be warm. In some embodiments, the rinse is performed by dispensing the adipose tissue into centrifuge tubes. Sterile water is added to the centrifuge tubes in an approximately 1:1 ratio of solution to tissue. The centrifuge tubes are shaken gently for approximately 2, 5, 10, 15 or 20 seconds. The tubes are then centrifuged at ambient temperature at a speed of 900-3655 RCF. In some embodiments, the rinse may be performed by dispensing the adipose into a sealable container. Sterile water is added to the sealable container in an approximately 1:1 ratio of solution to tissue. The sealable container is shaken gently a few times during rinse time. After the rinse, the tissue may then be decanted through a sieve, and any decanted liquid, oil or cell pellet is discarded. The sterile water rinse may be repeated multiple times.

Step 104e comprises removing excess water from the adipose tissue. In some embodiments, excess water is removed by loading a drying sieve into centrifuge tubes. A wipe is cut about the circumference of the drying sieve and placed on top of the sieve. The adipose tissue can be dispensed in equal amounts into the tubes and the tubes may be centrifuged at ambient temperature. The tubes may be centrifuged at a speed of 900-3655 RCF. The excess water may be drained through the bottom opening of the centrifuge tube. Any unwanted liquid drained may be discarded. Step 104e may be repeated for multiple centrifugal cycles (e.g. 2, 3, 4, 5 or more times).

Step 105 comprises pre-packaging the resulting adipose tissue allograft. Pre-packaging comprises combining the tissue filler product and gently mixing until a uniform consistency is achieved. The adipose tissue product may be prepared for packaging by dispensing the product equally into centrifuge tubes. The tubes may be centrifuged for approximately at a speed of 900-3655 RCF for a predetermined amount of time (e.g. 200, 300, 400, 500, 600, 800 or 1,000 seconds).

With reference now to FIG. 1C, a method for processing adipose tissue 150 comprises the steps of freezing and thawing a human adipose tissue 152, separating the human adipose tissue into pieces by mechanical grinding 154, serial rinsing the human adipose tissue with a non-ionic surfactant, a sterile crystalloid solution and sterile water 156, and dispensing the human adipose tissue into packaging using a loading tool 158. In one embodiment, the method includes the step of debriding the human adipose tissue to remove extraneous or bruised tissue. In one embodiment, the method includes the step of rinsing the human adipose tissue with a non-ionic surfactant, sterile crystalloid solution, and sterile water by dispensing the human adipose tissue into centrifuge tubes and centrifuging the human adipose tissue. In one embodiment, the method includes the step of rinsing the human adipose tissue with a non-ionic surfactant, sterile crystalloid solution, and sterile water by dispensing the human adipose tissue into a sealable container and gently shaking the container. In one embodiment, the method includes the step of rinsing the tissue in a non-ionic surfactant multiple times. In one embodiment, the method includes the step of rinsing the tissue in a sterile crystalloid solution multiple times. In one embodiment, the method includes the step of rinsing the tissue in sterile water multiple times. In one embodiment, the method includes the step of drying the human adipose tissue in a centrifuge tube comprising a drying sieve. In one embodiment, the method includes the step of decanting the tissue after each rinse and removing any decanted liquid and cellular components. In one embodiment, the method includes the step of homogeneously dispensing the human adipose tissue in containers using the loading tool. In one embodiment, the method includes the step of packing the containers in a sterile pouch.

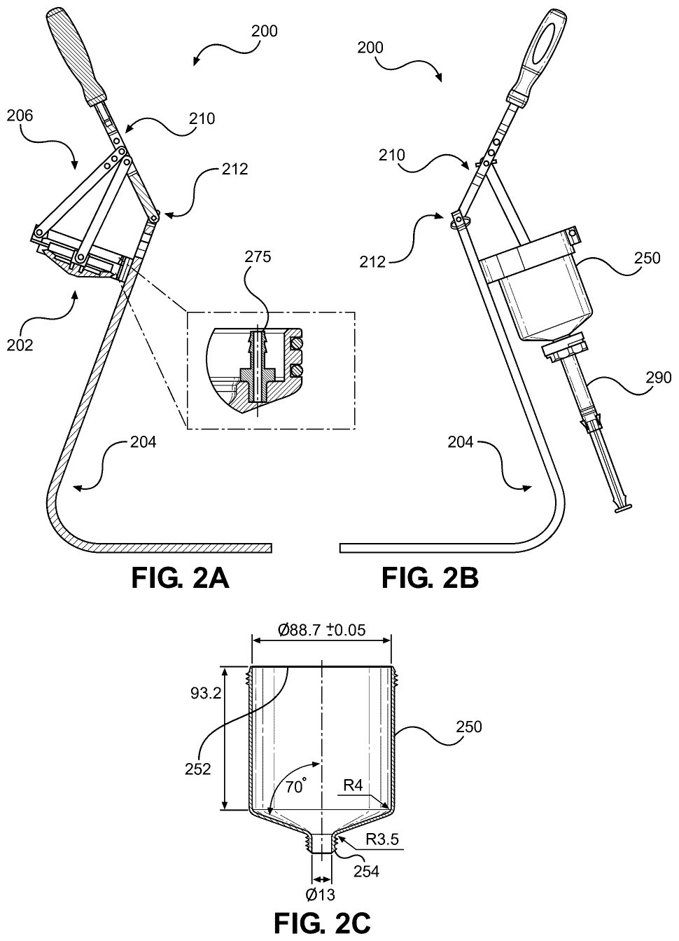

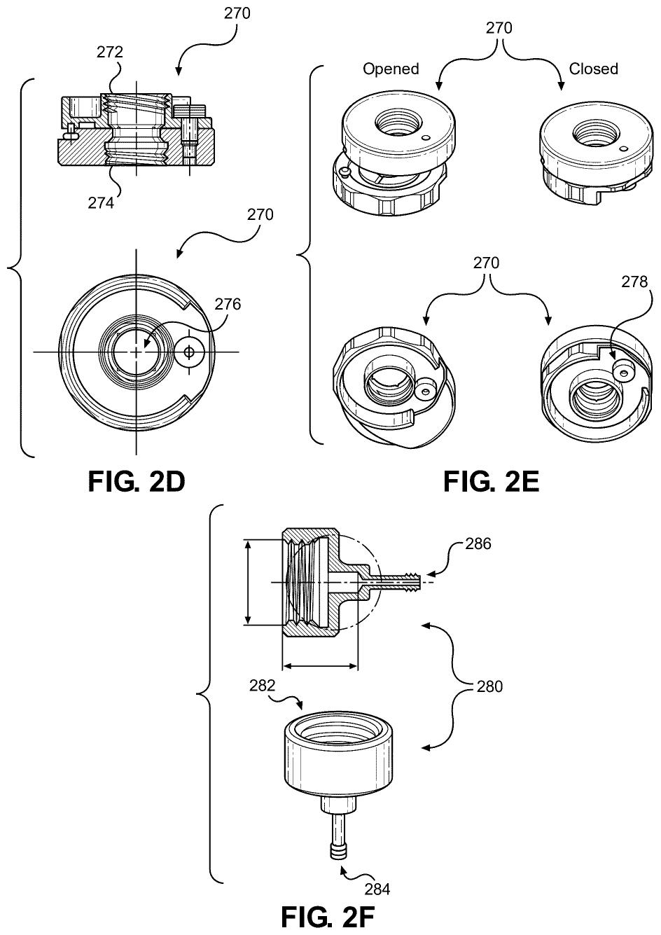

In some embodiments, the tissue product can be dispensed via a loading tool as shown in FIGS. 2A-2F according to one embodiment. The loading tool can be used in conjunction with the adipose tissue manufacturing process to enable the centrifugation of, atmospheric control, water content, compositional control, physical handling of, and dispensing of adipose allografts and similar tissues and media. The tool can be made to be sterilized via autoclave and requires no external electrical power. The loading tool can hold formable and flowable media during centrifugation, enclose the media during atmospheric transfer, remove unnecessary voids and gas pockets from the media, and dispense the media with adaptation to various different containers. In one embodiment, the loading tool 200 includes a jar 250 or container compatible with centrifugation with sealable openings at both ends, such as proximal opening 252 and distal opening 254. The sealable openings can for example include a threaded exterior for mating with a twist cap to seal the opening. The openings can be configured to enable the efficient decanting during liquid processing, then one way gas exchange during atmospheric control, then void free dispensing during dispensing. A first adapter 270 such as the one shown in FIGS. 2D and 2E (alternatively referred to as a container adapter) can mate with the distal opening 254 on the jar 250 via the threaded surfaces and provide a sealable access port 276 having proximal and distal openings 272, 274 to facilitate dispensing of the media from the Jar 250 into the container. A connecting structure 278 such as a threaded opening for a shoulder screw can be included for assembling separable parts of the first adapter 270. A second adapter 280 such as the one shown in FIG. 2F (alternatively referred to as a nitrogen adapter) can mate with the distal opening 254 on the jar 250 via the threaded surfaces and provide a sealable access port 286 having proximal and distal openings 282, 284 for fluid or gas communication with the jar 250, which for example can be used to remove gas or liquid from media in the jar 250. This sealable access port 286 may also be connectable to a valved conduit for creating sealed fluid communication.

The piston 202, stand 204, lock bar 206, jar 250, and nitrogen adapter 280 together facilitate the creation of an airtight container around the media. Ports on the nitrogen adapter and the piston 275 enable the one-way gas exchange to remove undesirable gasses such as oxygen from the media. The ports may for example have a controllable valve for controlling fluid and gas communication. The lever 210 and container adapter 270 work with the other components to dispense media in a void free and low oxygen manner. The lever 210 can be attached to the stand 204 at a hinged connection 212. A lock bar 206 can be added to arrest movement of the lever 210.

The loading tool can be fully disassembled and sterilized. The materials are compatible with various sterilization methods such as steam sterilization, Ethelye oxide, etc. The loading tool enables atmospheric control by sealed atmospheric exchange for product packaging. Containing the tissue in a sealed space with ports to facilitate the removal of naturally present atmosphere by vacuum and replacement in a through-flow fashion, backfilling with inert gas as desired. Atmospheric exchange enables preservation using various inert gases. The loading tool gently extrudes the tissue directly into a container with minimal atmospheric exposure, in a controlled fashion to create filled containers with minimal voids. The loading tool can be adapted with various tip configurations to be used with a desired container. Loading tool jars are also compatible with centrifugation both in shape, strength, and mass. A Jar compatible with centrifugation as previously noted with sealable openings at both top and bottom for the selectable removal of high density or low-density tissues, solutions, and/or debris. This along with the proximal and distal openings of the jar facilitate rinsing of the product and allow the use of the same container for product filling/packaging. Loading tool enables the water content control by suspended centrifugation. A Jar and drying sieve assembly that suspends tissue with or without the addition of a permeable membrane that suspends the tissue over an empty space during centrifugation to remove excess water and/or other solutions present in the tissue. Because the components are able to be autoclaved, the tool and its components are reusable.

The use of the loading tool for the packaging process allows the allograft to be efficiently dispensed in the primary packaging, which can improve shelf stability, cosmetic performance of the allograft, and enables controlled dispensing at the point of use. The loading tool is prepared for use by loading the centrifuge jar containing the adipose tissue onto an adapter. The loading tool's piston is slightly depressed to engage the O-ring, and the lock bar is inserted and latched. The nitrogen adapter is attached to the base of the jar, and the vacuum is attached to the piston.

The tissue is then purged of oxygen using the loading tool. The vacuum pump is adjusted (e.g. approximately −5, −10, −15, −20 Hg or more), and the vacuum line valve is opened. The nitrogen line valve is opened enough to reduce the vacuum (e.g. to approximately −5, −10, −15 Hg for 5, 10 or 15 seconds). The nitrogen line valve is turned off and opened a second time to reduce the vacuum for a second time. The nitrogen line valve is turned off, and then opened a third time and left on. The vacuum pump is turned off, while leaving the vacuum line open and allowing the nitrogen to bring the pressure to 0 in Hg. The nitrogen line valve is turned off.

The loading tool is then prepared for packaging. The lock bar is removed after the pressure normalizes and the piston is lowered to make contact with the adipose tissue. The vacuum line valve is closed and the nitrogen adapter is removed. The container adapter is attached with a container.

The tissue can now be packaged. The loading tool 200 is used in combination with the container adapter 270 to package tissue into a final primary container. As noted herein, although a syringe 290 is being referenced according to several embodiments, more generally several types of containers can be adapted for use, such as syringes, cannulas or other types of containers.

Step 106 comprises performing several quality control inspections to make sure the adipose tissue allograft is up to standard.

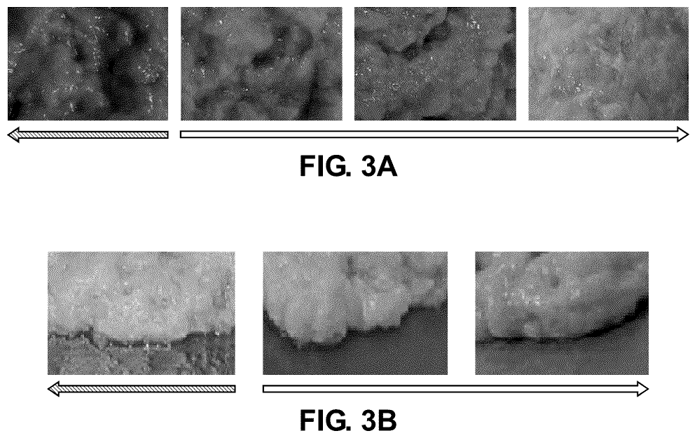

In some embodiments, the tissue is visually inspected for color. The product passes the inspection if the tissue color is off-white and/or has various tints/tones of yellow in color in accordance with the chart depicted in FIG. 3A. The product fails the inspection if the tissue is not off-white or various tints/tones of yellow in color in accordance with the chart as shown in FIG. 3A. If tissue fails the color inspection test, rework must be performed.

Rework comprises dispensing the adipose tissue equally into centrifuge tubes and submerging the adipose tissue in sterile 0.9% saline to approximately a 1:1 ratio of saline to adipose tissue. The centrifuge tubes are gently shaken for approximately 2, 5, 10, 15 or 20 seconds to disperse the adipose tissue into the saline. The centrifuge tubes are then centrifuged for 5 minutes at 900-3655 RCF. The tissue is then decanted through a sieve and any cell pellet, liquid or oil may be discarded. This process is repeated, and the adipose tissue is gently mixed inside the centrifuge tube and placed back in the centrifuge. The tissue is centrifuged for 3 minutes at 900-3655 RCF. The tissue is decanted through a sieve and any unwanted liquid or cellular components may be discarded. The visual color inspection is repeated. Rework may be repeated as many times as necessary until the product is acceptable. The final visual color inspection is recorded.

In some embodiments, a syringeablity test is performed. A full syringe is loaded with product (approximately 14 cc). The syringe is connected to a single exit cannula. The product is expressed through the cannula. The product passes the syringeability test if the entire contents of the syringe pass through the cannula using finger applied force. The product fails the syringeability test if tissue cannot be expressed using finger applied force on the plunger and/or the cannula gets clogged. Rework is performed if product fails the syringeability test, and the test is repeated.

Rework comprises expressing the tissue through a 3-4 mm sieve with a scraper into the bowl. Only the tissue that passes through the sieve is collected. The tissue is gently combined and mixed together until a uniform consistency is achieved. The adipose tissue is then dispensed equally into centrifuge tubes and submerged in sterile water to approximately a 1:1 ratio of sterile water to adipose tissue. The centrifuge tubes are gently shaken for approximately 2, 5, 10, 15, 20 seconds or more to disperse the adipose tissue into the sterile water. The tubes are then centrifuged (e.g. 200, 300, 400, 500, 600, 800, 1,000 seconds or more) at 900-3655 RCF. The adipose tissue is decanted through a sieve and any cell pellet, liquid or oil is discarded. The adipose tissue is gently mixed in the centrifuge tubes and placed back in the centrifuge and centrifuged (e.g. 60, 90, 120, 150, 180 seconds or more) at 900-3655 RCF. The tissue is again decanted into a sieve and any cell pellet, liquid or oil is discarded. The syringeability test is repeated and rework may be repeated as many times as necessary until the product is acceptable. The final syringeability test is recorded.

In some embodiments, the product is visually inspected for excess free lipid according to the chart depicted in FIG. 3B. The tissue is acceptable if the free lipid does not form an oily routine at or around the tissue in accordance with the chart. The tissue is unacceptable, and thus fails the test, if the free lipids form an oily outline at or around the tissue in accordance with the chart. If the product fails, rework is performed, and the test is repeated.

Rework comprises dispensing the adipose tissue equally into centrifuge tubes and submerging the tissue in sterile water to approximately a 1:1 ratio of sterile water to adipose tissue. The tubes are gently shaken (e.g. 5, 10, 15, 20, 25 seconds or more) to disperse the adipose tissue into the water. The tubes are then centrifuged for 5 minutes at 900-3655 RCF. The tissue is decanted through a sieve and any cell pellet, liquid or oil is discarded. The adipose tissue is gently mixed inside the centrifuge tube, placed back in the centrifuge, and centrifuged at 900-3655 RCF. The tissue is again decanted, and any cell pellet, liquid or oil is discarded. The free lipid test is repeated, and rework may be repeated as many times as necessary until product is acceptable. The final inspection result is recorded.

In some embodiments, an absorption measurement test is performed. An absorbing paper is placed on a clean, flat, nonporous surface. A pea-sized product sample is placed on absorbing paper. The tissue is allowed to settle for 15 minutes. Using a ruler, the travel distance from the point in which the tissue contacts the paper to the furthest edge of the lipid trace is measured. The product is acceptable if the lip trace travelled is less than or equal to 5 mm on the absorbing paper. The product is unacceptable if the lipid trace traveled is more than 5 mm on the absorbing paper. If the product fails the absorption measurement test, rework is performed, and the test is repeated. Rework involves dispensing the adipose tissue equally into centrifuge tubes and submerging the tissue in sterile water to approximately a 1:1 ratio of sterile water to adipose tissue. The tubes are gently shaken for approximately 2, 5, 10, 15 or 20 seconds to disperse the adipose tissue into the water. The tubes are then centrifuged for 5 minutes at 900-3655 RCF. The tissue is decanted through a sieve and any cell pellet, liquid or oil is discarded. The adipose tissue is gently mixed inside the centrifuge tube, placed back in the centrifuge, and centrifuged for 3 minutes at 900-3655 RCF. The tissue is again decanted, and any cell pellet, liquid or oil is discarded. The visual inspection and absorption measurement test is repeated and may be repeated as needed until the product is acceptable. The final test result is recorded.

Step 107 comprises packaging the product. Packaging comprises loading the product into containers using the loading tool. The loading tool is fully set up, and the tissue product is loaded into the containers using piston on the loading tool to reverse fill each container. The container is capped. A torque wrench may be used to tighten each cap. The loading step is repeated until all the product has been packaged into containers. All containers may be inspected for conformance with product specification, cosmetic quality, air pockets, and completeness of fill.

In some embodiments, each container is packaged inside a pouch. The pouch may be a foil or a foil chevron style pouch. Each container may be packaged such that the cap faces the chevron and away from the opening of the pouch. Each pouch may be vacuum sealed. Each seal may be inspected to ensure it is uniform, not wrinkled, and complete across the width of the pouch. A unique allograft ID label number may be applied to each pouch.

In some embodiments, each pouch may be packaged inside a pouch with a larger width and length. The internal pouch may be vacuum sealed. A single sealed pouch with the syringe inside may be placed inside the larger pouch. In some embodiments, the larger pouch may be a polyester or Tyvek chevron style pouch. The smaller pouch may be placed inside the larger pouch such that the chevrons are aligned. Each larger pouch may be heat sealed. Each seal may be inspected to ensure it is uniform, not wrinkled, and complete across the width of the pouch.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Experimental Example 1

Soft tissue defects can lead to a loss of normal tissue function, emotional trauma, and pain. There is a need for aesthetic and reconstructive surgical procedures, however, issues with current procedures exist such as varying success with volume retention, induction of angiogenesis, and complete wound healing. Minimally manipulated adipose products (MMAP) provide a non-surgical method in aesthetic or reconstructive surgical procedures that may overcome these issues. In the study conducted here, the safety, biocompatibility, and volume retention of the invention: a minimally manipulated adipose filler allograft, hereinafter referred to as “allograft,” was evaluated. Results demonstrated that the allograft is biocompatible with human systems as observed in vitro through suppressed T-cell proliferation and viability and in vivo where the allograft was implanted in athymic mice for 3 months and no significant immune response or safety risk was observed. Further, in vivo allograft implants demonstrated full wound closure and all mice remained in good health for the entire duration of the study. Allograft implants retained matrix and had observed increased vascularization and local fat organization as observed by histopathology. Finally, volume retention of the allograft was observed in vivo with high volume implantation (200 μl) maintaining visible implant volume for up to 3 months. Our findings show that the allograft is a versatile MMAP that synergistically integrates with surrounding tissues making a potential off-the-shelf product for use in aesthetic or reconstructive surgical procedures.

The study presents a new human MMAP adipose tissue allograft, as an excellent volumetric filler candidate for aesthetic or trauma-based soft tissue supplementation. The response from the allograft's natural yellow color and ease of handling for implantation, including its bulk and flowability, allows for various applications and methods of delivery, e.g. surgical implantation or injection. No adverse immune responses were reported in both in vitro and in vivo studies. None of the mice implanted with either low or high implant volumes suffered any health issues and remained in good health for the entire duration of the study, 3-months. The high-volume implant maintained better volume retention and was firmer when removed but presented signs of tissue encapsulation and potential preliminary signs of fat necrosis, while the low-volume implant resorbed much quicker into the native tissue. Finally, the implants were remodeled, and evidence of collagen formation promotion, increased vascularization, and changes in local fat organization were observed by histopathologist. Our findings show that the allograft is a versatile human MMAP that synergistically interacts with surrounding tissues making a potential off-the-shelf product for surgical use.

### Experimental Example 2

This study evaluated the compositional characterization of the Adipose Allograft. Adipose tissue is a connective tissue composed of clusters of cells (adipocytes) surrounding a thick extracellular matrix (ECM) (Mariman, E. C., et al., 2010. Cellular and molecular life sciences, 67(8), 1277-129.), among other cells, including preadipocytes, fibroblasts, vascular endothelial cells, and macrophages (FDA Guidance, Regulatory Considerations for Human Cells, Tissues, and Cellular and Tissue-Based Products: Minimal Manipulation and Homologous Use, July 2020). Adipose tissue provides cushioning and support for the skin among other tissues, stores energy in the form of lipids, and insulates the body, among other functions (FDA Guidance, Regulatory Considerations for Human Cells, Tissues, and Cellular and Tissue-Based Products: Minimal Manipulation and Homologous Use, July 2020), like expression and secretion of factors and proteins with important endocrine functions, like adiponectin, TNFα, IL-6, and other cytokines (Kershaw, E. E. et al., 2004. The Journal of Clinical Endocrinology & Metabolism, 89(6), 2548-2556.) Adipose Allograft invention retains adipose tissue's intrinsic natural characteristics, that is adipocyte structure and extracellular matrix.

The materials and methods of this experiment are now described. Adipose Allograft samples were analyzed through multiple methods to characterize its composition. Adipose Allograft samples were subject to DNA quantification through a DNA assay kit. Quantifying DNA content of samples supports the efficacy of reducing DNA content following the processing methods used to generate Adipose Allograft. DNA assay consists of extracting and isolating DNA from samples by using a series of buffers, then purifying it by using a spin column. Isolating DNA is then quantified using a spectrophotometer.

Adipose Allograft samples, a previous product iteration the adipose allograft, and unprocessed adipose samples (also referred to as raw) were subject to a compositional analysis that determined the major components of the samples, specifically water, lipid and solid (including ECM), that were quantifiable on a weight by weight (in grams) percentage basis. The results were compared.

Adipose Allograft samples and raw samples were evaluated to identify their structural protein content (including ECM) and protein retention in allografts as compared to native tissue through proteomics by a third-party vendor. Proteomic studies serve to identify proteins in a sample and confirm their presence through mass spectrometry.

Adipose Allograft samples and raw samples were processed for histology, embedded in paraffin, cut, stained, and imaged by a third-party vendor. Images of tissue samples presenting their microscopic structure were generated to compare the Adipose Allograft samples to unprocessed samples and support if structural components were retained in allograft as compared to native tissue. Samples were stained with Masson's Trichrome and Hematoxylin and Eosin (H&E). Masson's Trichrome allows visualization of connective tissues (including ECM) in samples. H&E allows visualization of tissue structure and DNA in the nuclei of adipocytes.

The results of the experiments are now described. During the development of the Adipose Allograft, it was imperative that the processing included a series of steps to improve reduction of cellular and allogenic components through decellularization methods while concurrently leaving behind the structural components, like extracellular matrix (ECM). Decellularization is the process of removing any allogenic or xenogeneic cellular antigens from tissue that would induce an immune response (Bruyneel AAN, et al., Artif Organs. 2017). Inadequate reduction of cellular and allogeneic components may trigger the innate immune system, which is part of the immune system that is nonspecific and attacks any substance that is deemed foreign. This may lead to inflammation and if severe enough, rejection of a graft (Kasravi M, et al., Biomater Res. 2023). Crapo et al. stated decellularization techniques cannot remove 100% of cell material (Crapo P M, et al. Biomaterials. 2011). Therefore, the development of the Adipose Allograft had a focus on reduction of cellular and allogenic components in the final product as compared to the native tissue.

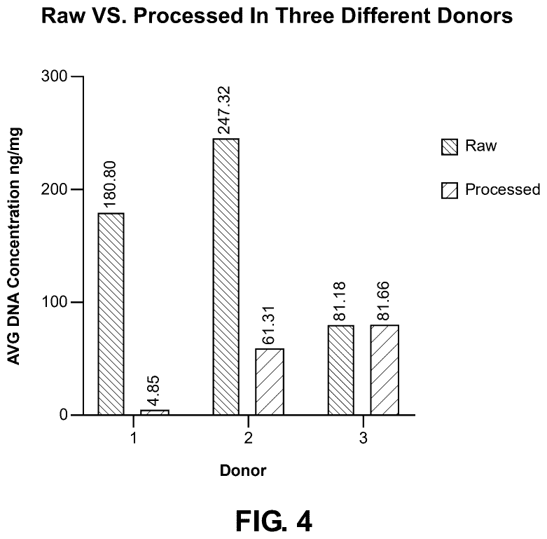

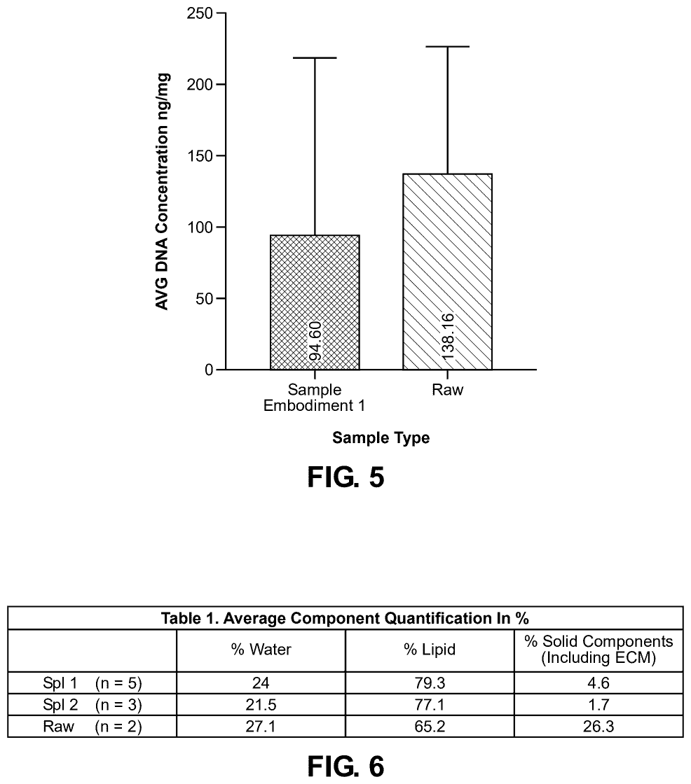

FIG. 4 shows the average DNA concentrations among three donors, both raw and Adipose Allograft samples. Results show lower DNA concentrations in the Adipose Allograft as compared to raw. Overall, the Adipose Allograft samples contained less DNA than its raw counterpart as supported by FIG. 5. FIG. 4 also depicts sample variability. One challenge in obtaining a staple number of DNA quantity per sample is donor variability. Each donor has unique characteristics that can lead to an inconsistency of data as seen in FIG. 6.

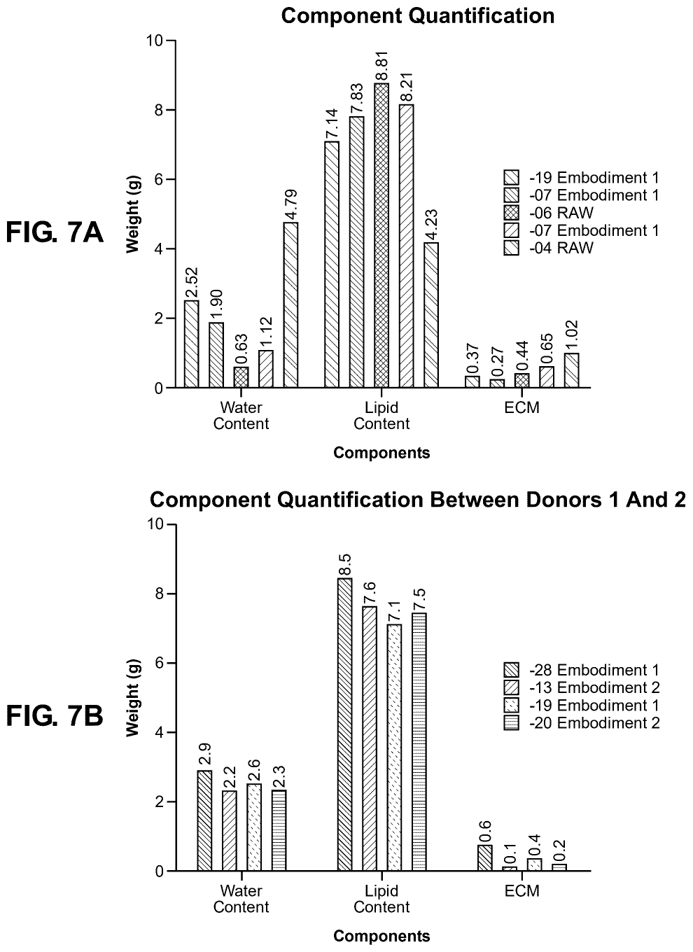

During the development of Adipose Allograft, it was helpful to understand the compositional characterization of Adipose Allograft to accurately describe the product and compare to native tissue or other grafts. A compositional analysis was developed to determine the major components of adipose tissue-based samples specifically water, lipid, and solid (including ECM) components, for comparison purposes. The resulting sample component amount averages in each respective form of adipose tissue-based samples, that is first and second sample embodiments and raw, are presented on FIG. 6, FIG. 7A and FIG. 7B. Variability among samples of the same type was observed, which may be attributed to donor and sample variability as each donor is unique in composition and sample homogenization may not always be achieved. The results seem to indicate a relationship between solid components and water content. This could be due to the proteins in the ECM being hydrophilic, which favors attachment (Yang, L. et al., RSC Advances, 2017). On average, the percentage of solid components (including ECM) was increased in Embodiment 1 samples as compared to Embodiment 2. In future studies, it is recommended to increase the number of samples analyzed to increase confidence in results.

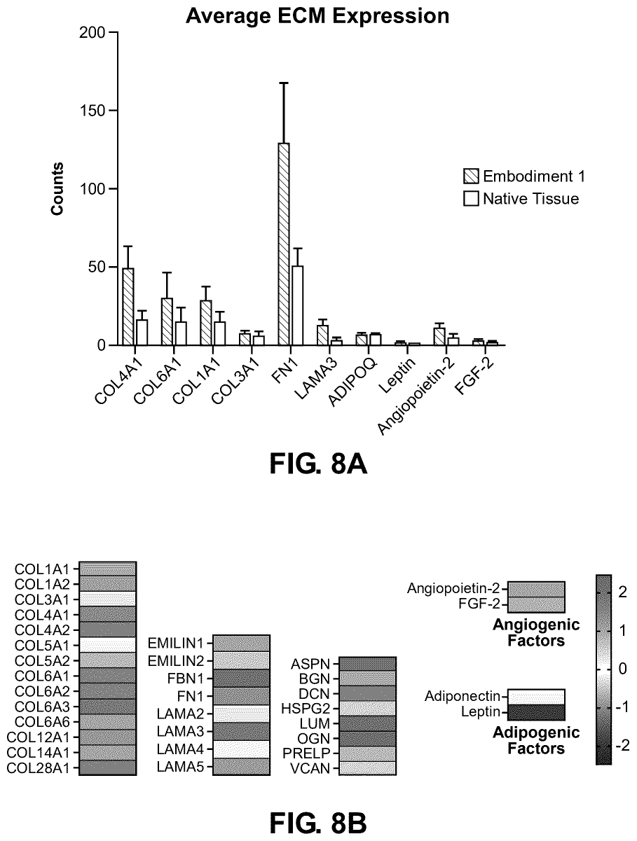

During the development of the Adipose Allograft processing, a non-ionic surfactant was used as a mild detergent to reduce cellular and allogenic components while avoiding degeneration and wash-out of structural proteins. Proteomic studies were used to confirm the presence of structural proteins in the Adipose Allograft as compared to native adipose tissue. As shown on FIG. 8A and FIG. 8B, the following proteins were retained in four distinct allograft samples as compared to the native tissues from the same donor. The Adipose Allograft retains major adipose collagens (COL4, COL6) as well as major fibrillar (COL1, COL3) and minor fibrillar (COL5) collagens. These proteins are responsible for the formation of the ECM and are some of the most abundant proteins in adipose tissue (Mori S, et al. Int J Biol Sci. 2014., Ricard-Blum S. Cold Spring Harb Perspect Biol. 2011). The Adipose Allograft also retains glycoproteins, key adipose ECM proteins fibronectin and laminin-α, as well as emillin1,2, adiponectin (associated with adipocyte proliferation) and fibrillin1 (contributes to adipocyte development) (Ricard-Blum S. Cold Spring Harb Perspect Biol. 2011). Further, the Adipose Allograft retains various proteoglycans. A key component in proteoglycans is glycosaminoglycans (GAGs) which is a main ECM protein in adipose; also responsible for basement membrane development, water retention, and transporting growth factors across the ECM (Pessentheiner A R, et al. 2020).

During the development of the Adipose Allograft, the graft is prepared though minimally manipulative processing methods that maintain the structure of adipose tissue in the graft as in native tissue. Histological images of tissue samples presenting their microscopic structure were collected to confirm the adipose tissue structure is retained by comparing images of native adipose tissue to images of the Adipose Allograft. Tissue samples were prepared with two distinct stains, Massons's trichrome and H&E to observe the tissue's microscopic structural composition. Masson's Trichrome is a beneficial stain to employ because it targets connective tissue like collagen, keratin, fibrin, and other structural components (Zhou X, et al. Bio Protoc. 2017). Masson's Trichome stain is a three-color stain that identifies connective tissue in blue/green color, cell nuclei in red/purple, cell cytoplasm in pink. H&E is a beneficial stain to employ because it targets cellular structure and nuclei (Zhou X, et al. Bio Protoc. 2017). H&E is a dual stain that identifies cell nuclei in a purple/blue stained by hematoxylin and cytoplasm is red/pink stained by eosin.

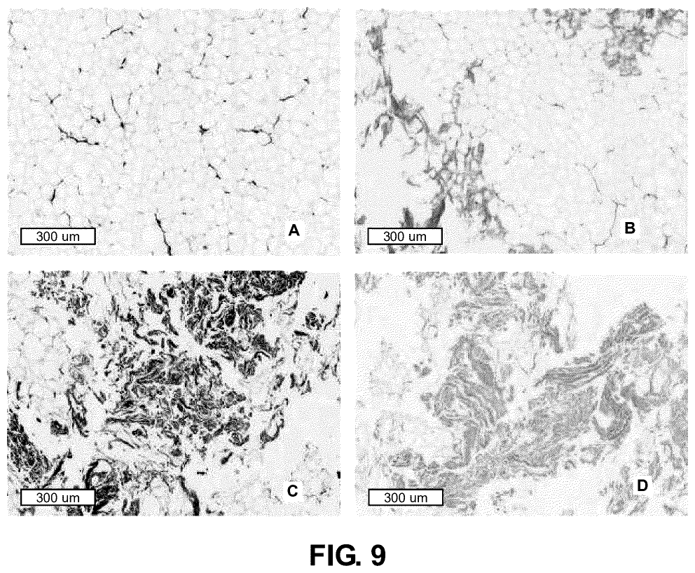

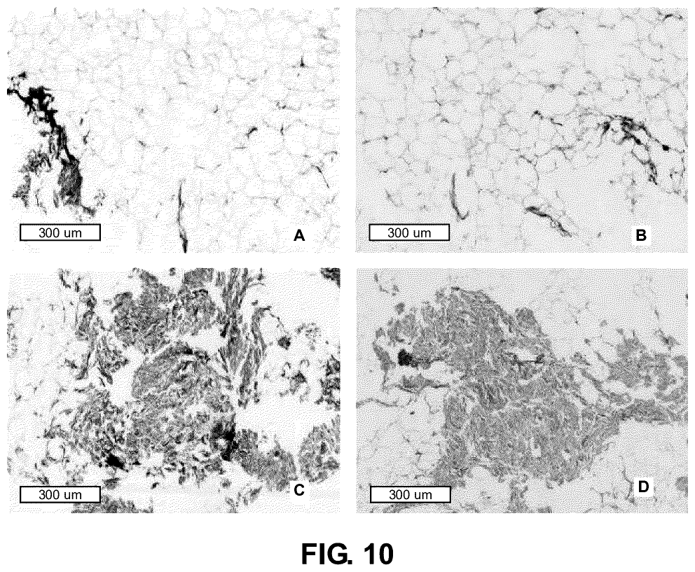

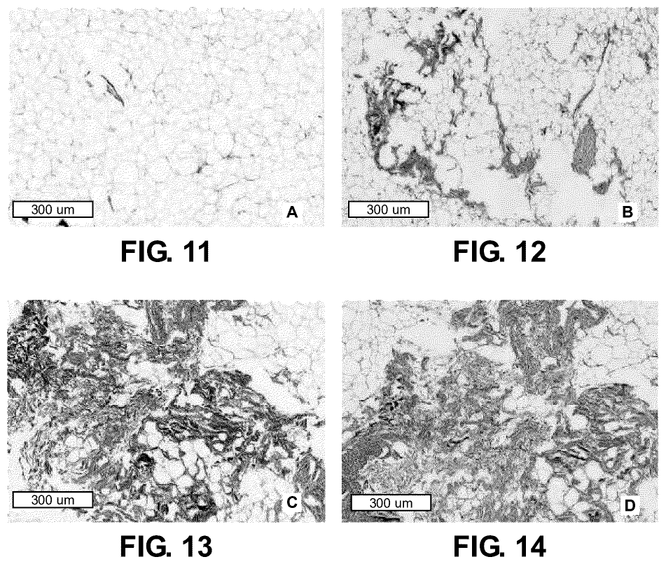

Histological images of tissue samples, depicted on FIGS. 9-14, show the Adipose allograft retained structural components, like adipose cell structure (represented by the honeycomb-like structure) and connective tissue network (including ECM) that comprises adipose tissue as compared to native tissue. This observation is consistent throughout the various samples. Histological images depicted in FIGS. 9-14 also show an increased amount of connective tissue (including ECM) staining compared to the individual adipose cell structure in the Adipose Allograft samples as compared to native tissue. This observation is consistent throughout the various samples.

### Experimental Example 3

This study evaluated the aging performed on adipose tissue-based research allografts in real-time. Real-time aging studies provide data to determine a product's shelf-life and the effects of aging on materials. During the study, the product sits on a shelf, and is exposed to environmental changes, such as temperature and humidity fluctuations, that simulate the real-world circumstances the product may experience during its lifecycle. In this study, the conditions of real-time aging were simulated to gather data on aging of adipose tissue-based grafts for research purposes only.

The materials and methods are now described. Human adipose tissue were designated for research with appropriate donor consent and permissions. The donors were tested and found negative for the following infectious diseases: antibodies to the human immunodeficiency virus (types 1 and 2), nuclear acid test (NAT) for HIV-1, hepatitis B surface antigen, hepatitis B core antigen antibody, antibodies to the hepatitis C virus, and syphilis. Pre-processing cultures of recovered tissue were obtained and found to be negative for the following pathogenic, highly virulent microorganisms Clostridium, fungi (yeasts, molds); and Streptococcus pyogenes. Adipose tissue was recovered in a manner to preclude contamination and/or cross-contamination.

Adipose tissue transportation, storage, processing, and packaging was performed and documented in accordance with the methods disclosed herein. Adipose tissue was processed in a manner to minimize contamination and/or cross-contamination. Grafts were subject to gamma radiation with a minimum dose of 15 kGy at a third-party contractor according to their procedures. A record of the radiation dosimetry was obtained. Following irradiation, grafts were transported at ambient temperature to a location designated for research with stable environmental ambient conditions. Grafts were stored at ambient temperature prior to evaluation. Evaluation time points were approximately months apart.

At least 1 graft unit was subject to bioburden testing prior to irradiation. Additional bioburden testing was performed after irradiation on at least 1 graft to confirm the lack of microbial contamination. A minimum of 1 graft unit was evaluated at selected time point. The tissue in each graft was allocated in a manner to maximize the number of evaluations. All evaluations were optional as they depended on graft availability. Quality Control Inspection was performed in-house. Grafts were prepared for histology for imaging to evaluate the structural characteristics of the product on a microscopic scale and potential changes over time. Mechanical testing was performed. In-Vitro Cell Survival Assay was performed in-house to evaluate if a test sample may affect cell survival, either NIH/3T3 or NCTC clone 929 (L cell, L-929, derivative of Strain L) mouse fibroblast cell lines.

The results of the experiments are now described. Bioburden testing was performed prior to irradiation on the Adipose Allograft research samples from 4 different donor lots. Bioburden testing was also performed following irradiation on the Adipose allograft research samples from 2 different donor lots.

Quality control inspections, histology, mechanical assays and in-vitro survival assays were performed throughout the study period to evaluate the structural integrity, free lipid content, cell survival and overall shelf life of the product. Bioburden testing prior to irradiation indicated contamination present in the sample of no greater than 14 CFU. Bioburden testing following irradiation supports lack of microbial contamination following irradiation with results of no growth. A total of 20 samples were evaluated for Quality Control inspection of tissue size at various time points from 0 to almost 6 months. All samples passed the evaluation at various time points. Results suggest that the products tissue size remains consistent over time up to almost 6 months.

A total of 22 samples were evaluated for Quality Control Inspection for color at various time points from 0 to almost 6 months. All samples passed the evaluation at various time points. Results suggest that the product's color is acceptable over time up to almost 6 months.

A total of 22 samples were evaluated for Quality Control Inspection for free lipid content at various time points from 0 to almost 6 months. All samples evaluated from 0 to 4 months passed the evaluation. Future studies may include evaluating triglyceride and glycerol content over time to quantify free lipid content and trend potential changes.

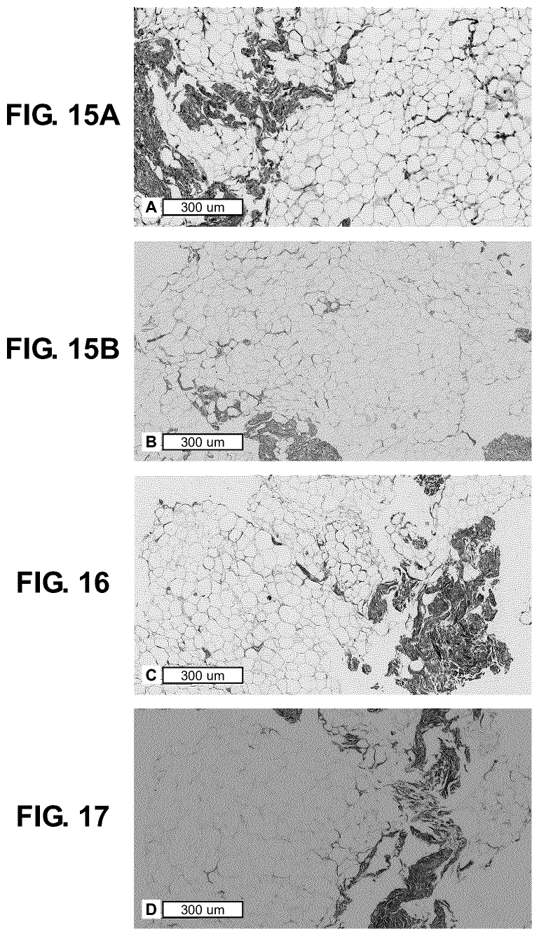

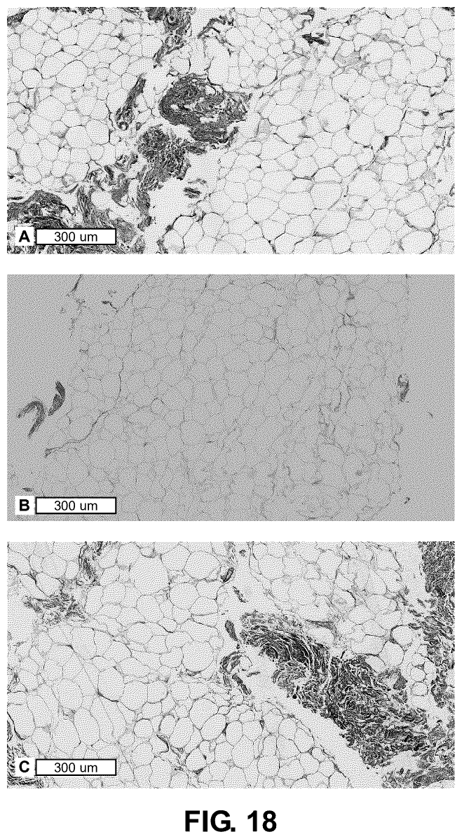

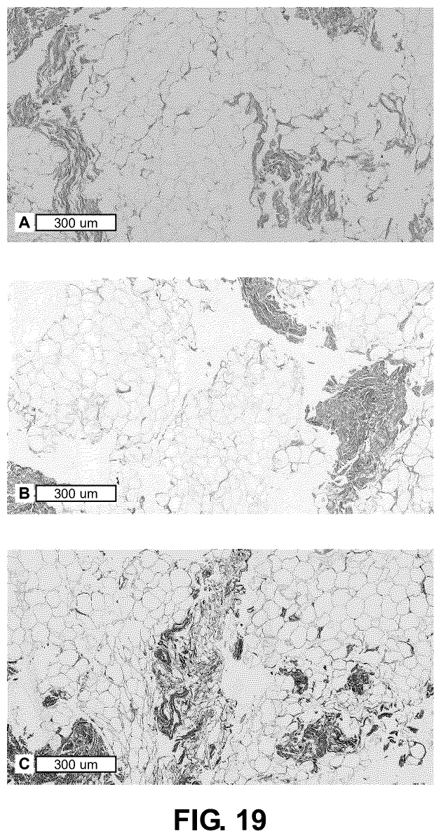

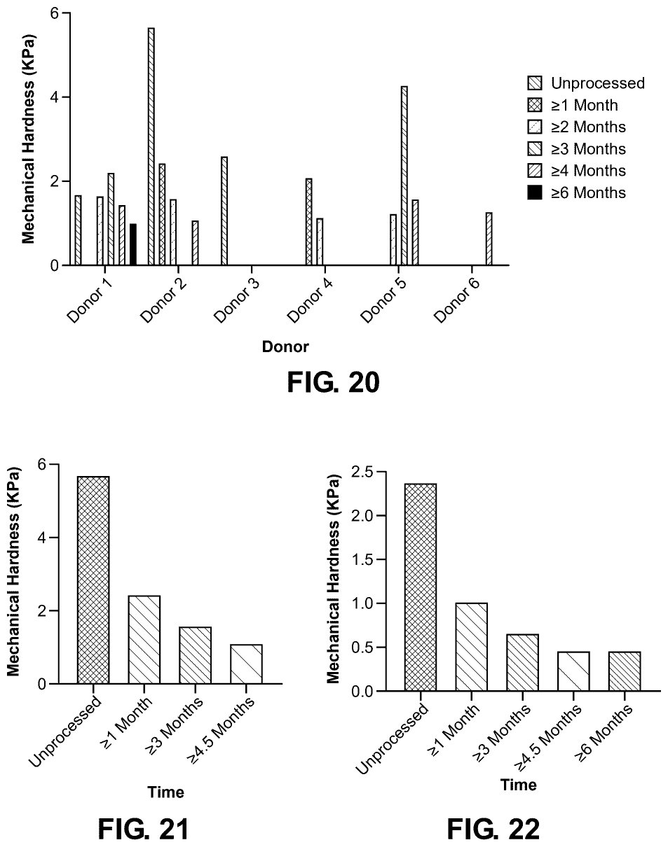

Adipocyte and connective tissue structure could be observed in histological images from samples stained with H&E and Masson's Trichrome over all time points. Histology was performed with the samples. This histological data suggests the Adipose Allograft samples retain the natural adipose structure over time. FIGS. 15-17 show images of Adipose Allograft samples stained with H&E from a single donor at time points of 2, 3, 4, 6 months. FIG. 18 shows images of Adipose Allograft samples stained from another donor at 2, 3 and 4.5 months. FIG. 19 shows images of Adipose Allograft samples stained from another donor at 1, 1.5 and 2.5 months. Adipocyte and connective tissue structure could be observed in most histological images from samples stained with Oil Red O over all time points. It was identified that histological images showing poor sample structure were prepared under the same order, which suggests poor sample preparation technique from the histology laboratory.

Mechanical Assay results revealed a slight downward trend in mechanical hardness of Adipose Allograft samples appears to be observed. The mechanical hardness of two distinct donor samples over time, along with an unprocessed sample. However, no conclusive statistical evidence supports the downward trend as the sample size per time point and donor was not large enough to perform statistical analysis.

A total of 8 samples were evaluated with the in-vitro cell survival assay at various time points from 0 to almost 6 months. In-vitro cell survival assay was performed with various samples. All samples passed the assay thus suggesting that fibroblasts cells' survival or growth were not negatively affected by the test samples up to almost 4 months. 4 months was selected as the maximum time point since at least 3 samples were assayed at this time point, compared to a single sample at 6 months.

### Experimental Example 4

This study evaluated the differentiation of adipose-derived stem cells (ASCs) to adipocytes and determined if the presence of a test sample of the tissue product altered the ASC differentiation properties via a control or other test sample.

Adipose-derived stem cells (ASCs) have raised interest in therapeutic applications for regenerative medicine due to the line's multiple attributes. ASCs have low immunogenicity and the ability to self-renew, can migrate into damaged sites, act through autocrine and paracrine pathways, along with being multipotent (can differentiate to adipocyte, osteoblast, and chondrocyte lineages).

The materials and methods are now described. ASCs were thawed from liquid nitrogen and prepared for seeding in T150 culture flasks. After T150 flasks reached 90% confluency, 6,579 cells/cm² were then subcultured into each well of a 24-well plate. Designated cells received control stem media with no testing sample, control stem media with testing sample added, differentiation media with no testing sample, differentiation media with testing sample added, conventional with control stem media, and conventional with differentiation media. Stem media was replaced every 2-3 days until cells in the wells designated as differentiation wells reached 85-90% confluency. After confluency was reached, control stem media was replaced with differentiation media with the same feeding frequency. Inserts with testing samples were added the day after seeding. Pictures were taken 24 hrs., 48 hrs., 1 week and 2 weeks after adding the insert. After two-week cells were examined to see if differentiation occurred. Cells were treated with a fixative and then stained with Oil Red O to observe formation of lipids. Oil Red O was then eluted and read through a spectrophotometer.

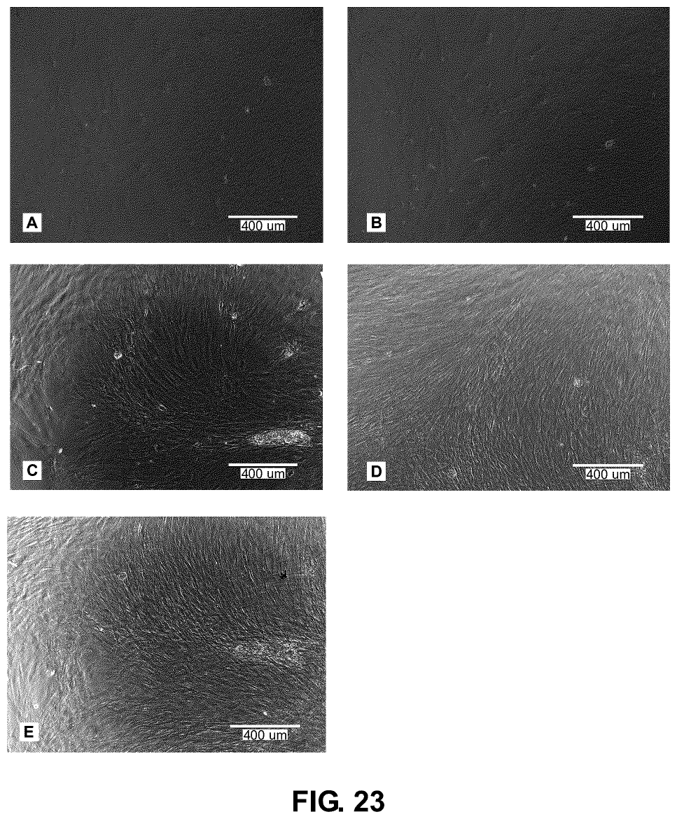

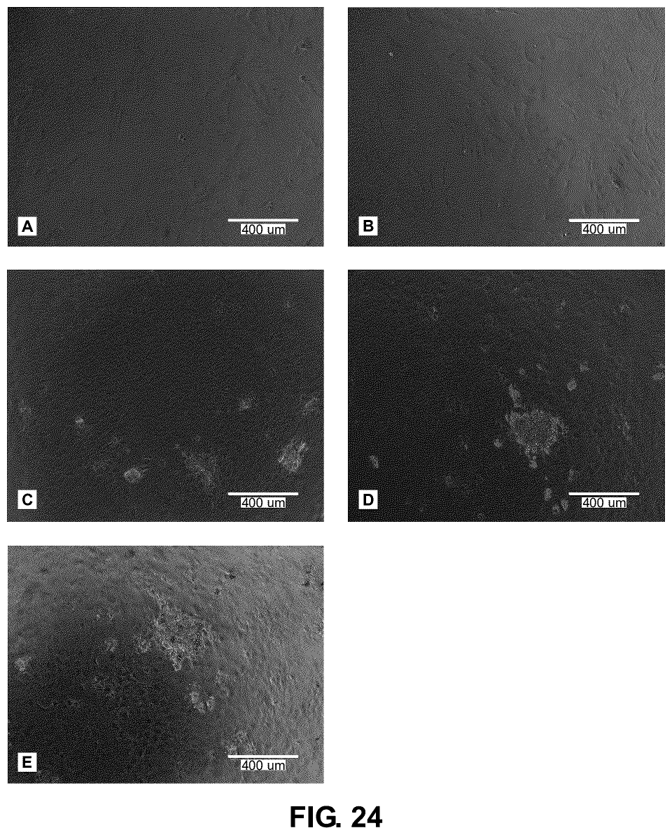

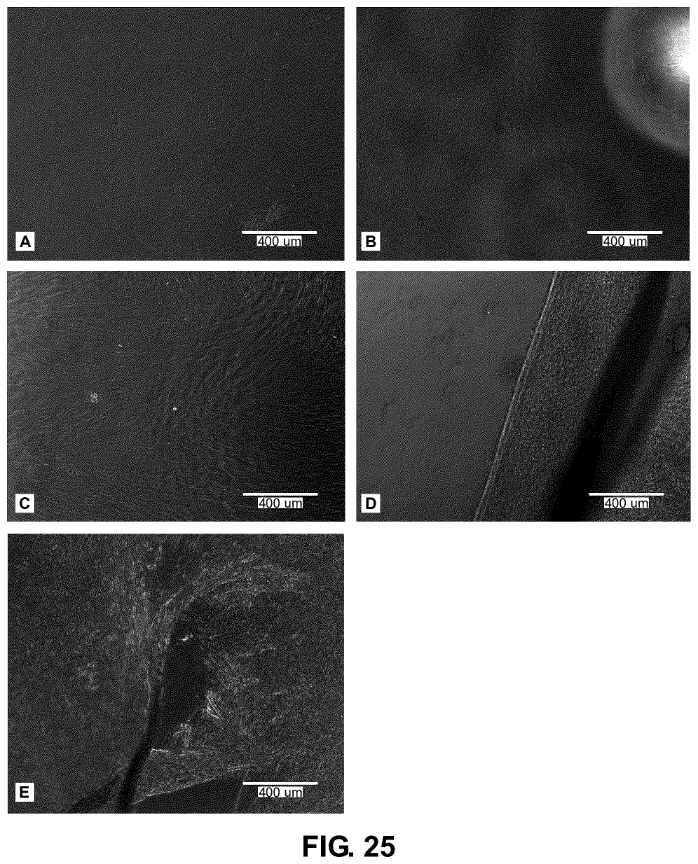

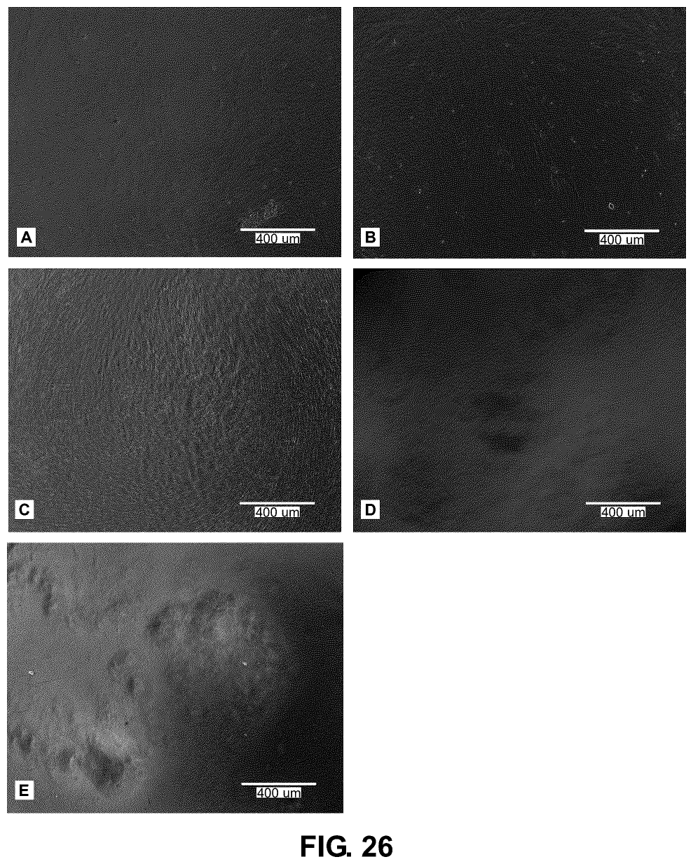

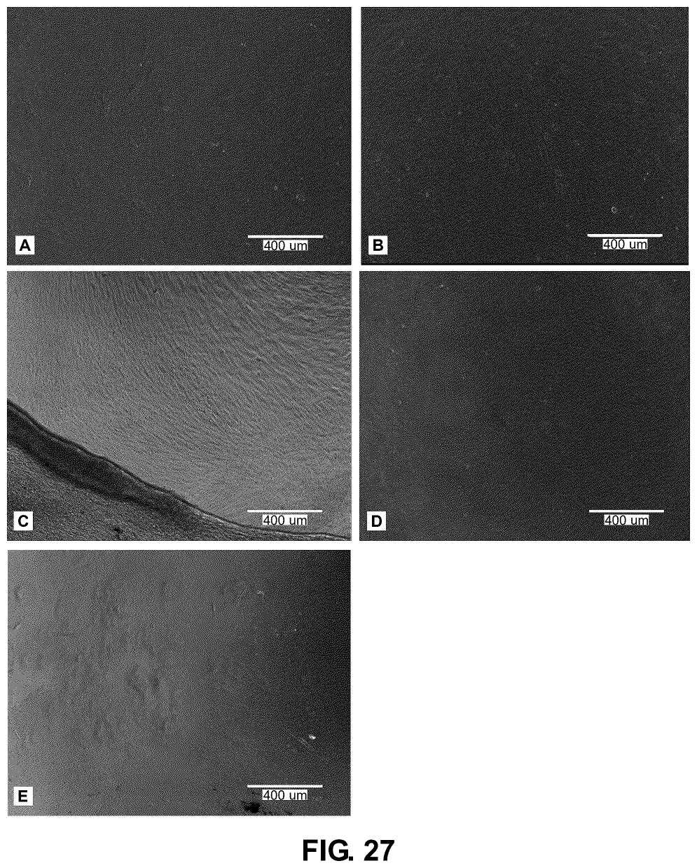

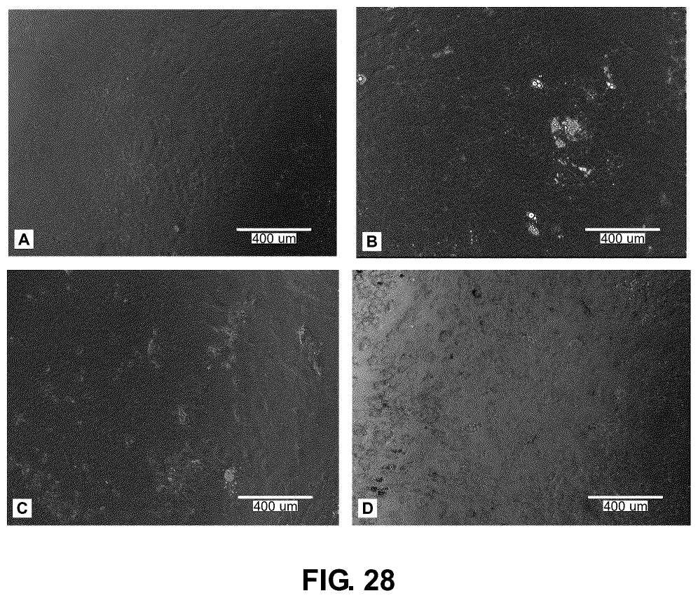

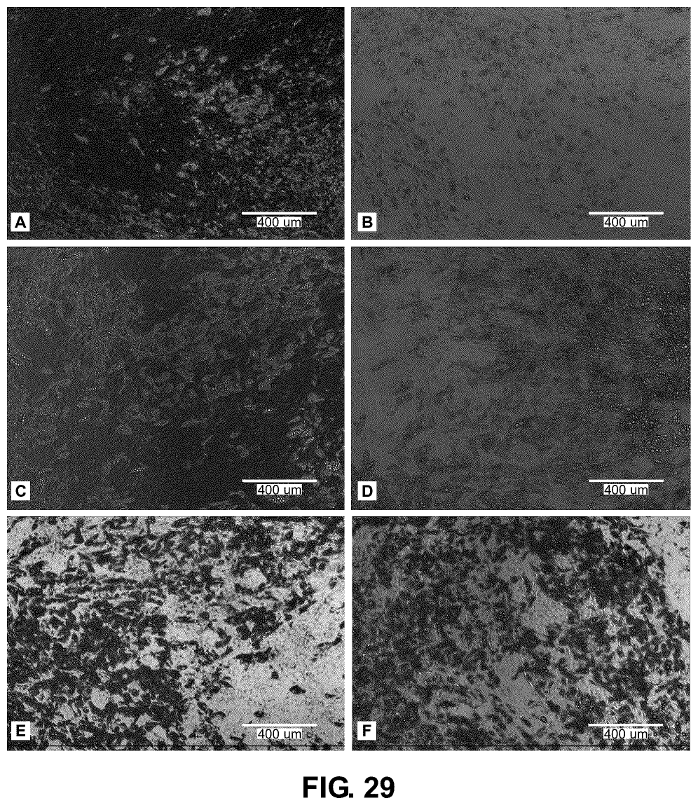

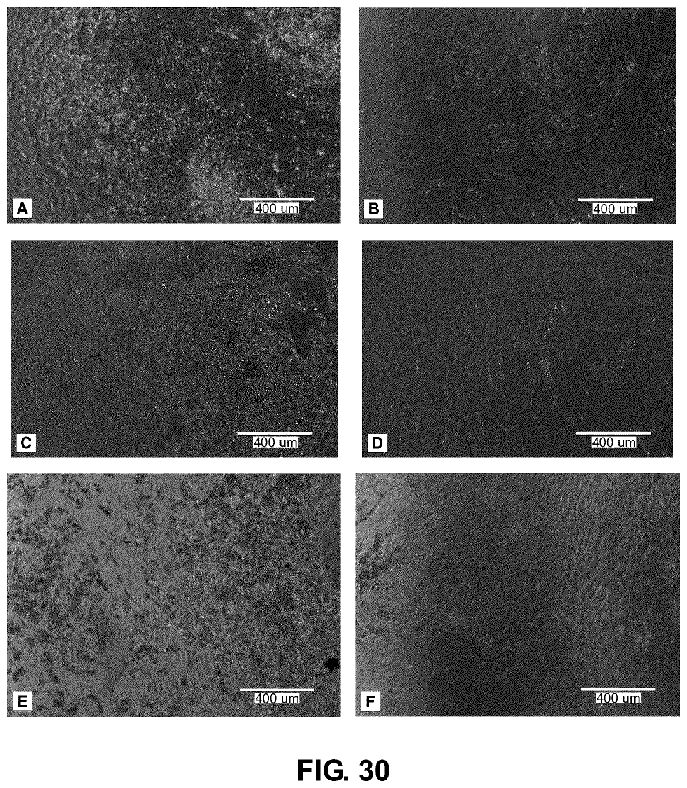

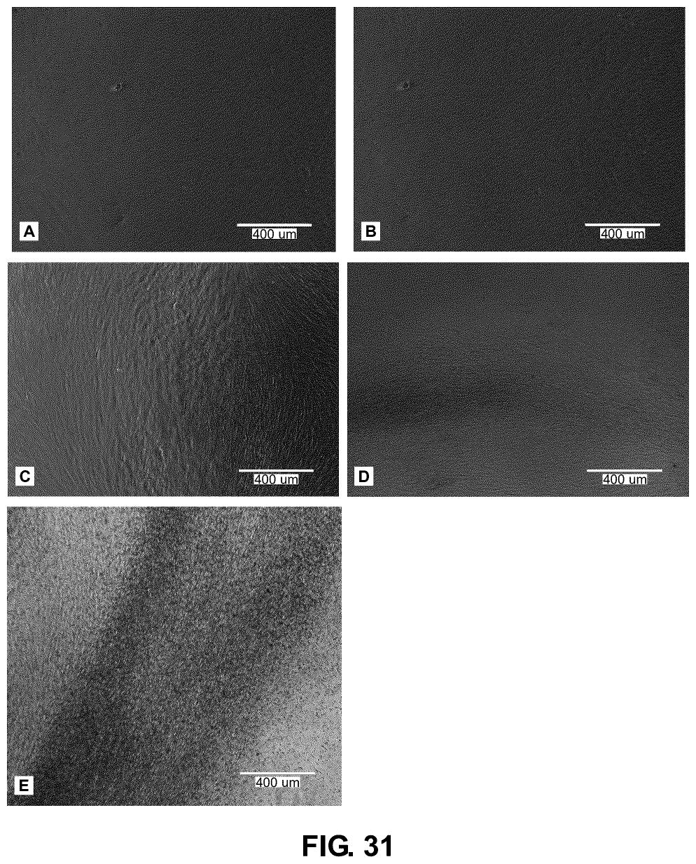

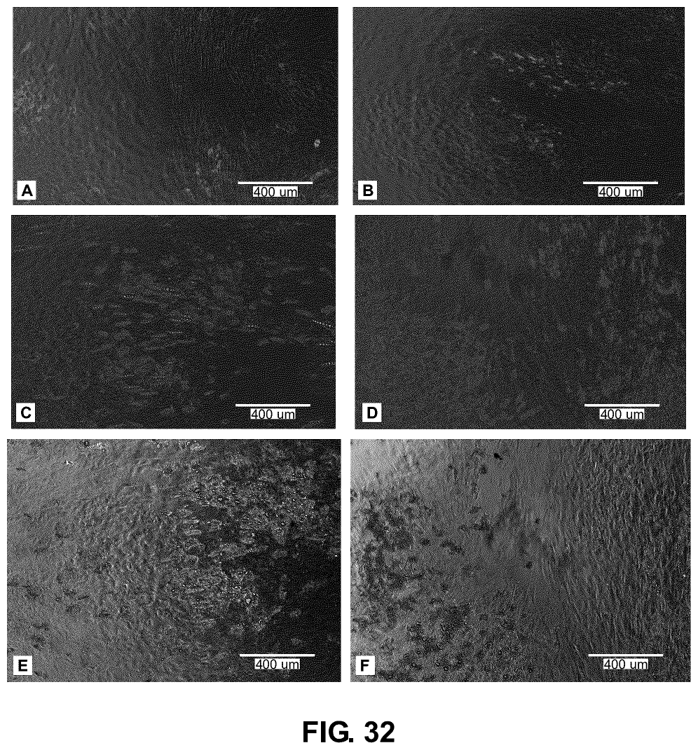

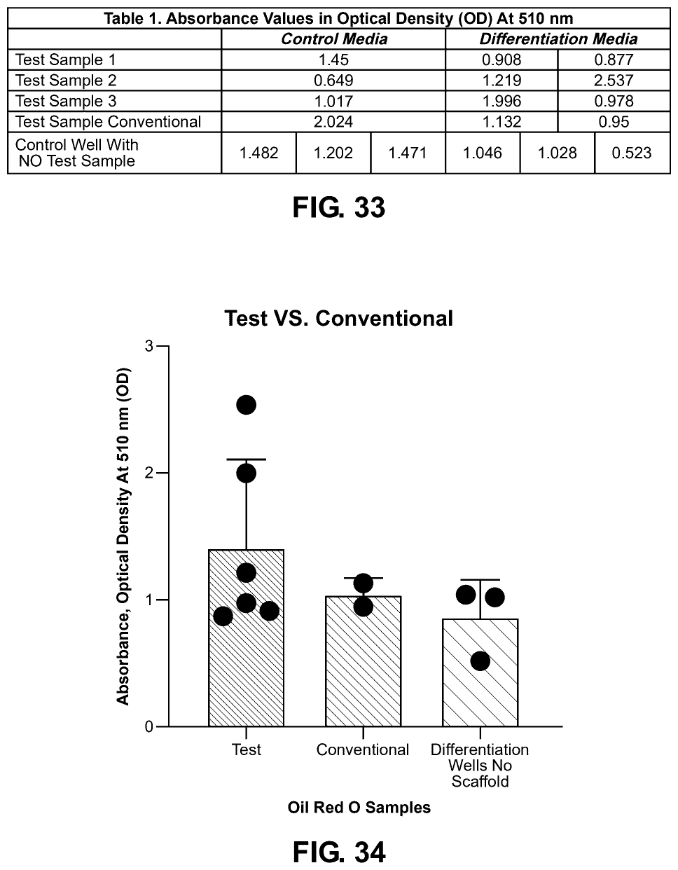

The results are now described. After two weeks in culture, the cells were stained with Oil Red O and evaluated for formation of lipid. FIG. 23-32 show results after 2 weeks. FIG. 23 cells in control media with no test samples added. FIG. 24 differentiation well with no test samples added (also a control well). FIG. 25 cells with control media and donor 1 test samples. FIG. 26 cells with control media and donor 2 test samples. FIG. 27 cells with control media and donor 3 test samples. FIG. 31 cells with control media and conventional test samples. FIG. 28 cells with diff media and donor 1 test samples. FIG. 29 cells with diff media and donor 2 test samples. FIG. 30 cells with diff media and donor 3 test samples. FIG. 32 cells with diff media and conventional samples. FIG. 33 and FIG. 34 present the mean absorbance as represented by Optical Density (OD) at 510 nm of Test samples of an embodiment of the invention vs Conventional using a spectrophotometric assay. Levels of OD correlated to the amount of Oil Red O stain in ASCs that differentiated into adipocytes. It was found that all ASC groups grown in Differentiation Media had substantial differentiation into adipocytes as shown by lipid formation identified by Oil Red O stains. No significant differences were observed in ASC differentiation into adipocytes between ASCs grown in the presence of test sample Adipose Allograft with Differentiation Media, mean OD 1.5±0.63 SD, and cells grown in Differentiation Media without test sample, mean OD 0.87±0.24 SD. No significant differences were observed in ASC differentiation into adipocytes between ASCs grown in the presence of test sample conventional with differentiation, mean OD 1.04±0.09 SD, and cells grown in Differentiation Media without test sample, mean OD 0.87±0.24 SD. The results also suggest ASC differentiation into adipocyte may be supported by the presence of Test sample Adipose Allograft and conventional, with slight better differentiation observed in the presence of the Adipose Allograft.

### Experimental Example 5

In this experiment, the trans-well paracrine effect of a test sample in the cellular proliferation of endothelial cells was determined. Primary Human Dermal Blood Endothelial Cells (HDBEC) are a subpopulation of the Human Dermal Endothelial Cells. They are isolated from the dermis of adult skin from a single donor. The cells are analyzed positive for CD31 and negative for podoplanin by flow cytometric analysis. Blood Endothelial cells have a key function in physiological processes like vessel tonus, capillary permeability, blood coagulation, fibrolysis, and angiogenesis. Primary HDBECs were selected for this assay due to their use in tissue engineering models (Pappalardo A et al., 2023. Sci Adv.)

The materials and methods are now described. To determine the effects on cellular proliferation, 7,895 cells/cm² primary HDBECs were seeded in 24-well plates grown in complete growth media and in the presence of test samples. After 3 days in culture, cells were stained with Crystal Violet (CV) and evaluated for changes in total cell number. Positive and negative control wells were set up to observe cellular proliferation of primary HDBEC in the absence of test sample using complete growth media vs basal media respectively. Cellular proliferation was expected from well with primary HDBEC grown in complete growth media, this establishing an assay positive control. Cellular proliferation was not expected from well with primary HDBEC grown in basal media, thus establishing an assay negative control. After 3 days in culture, cells were stained with CV and evaluated for changes in total cell number.

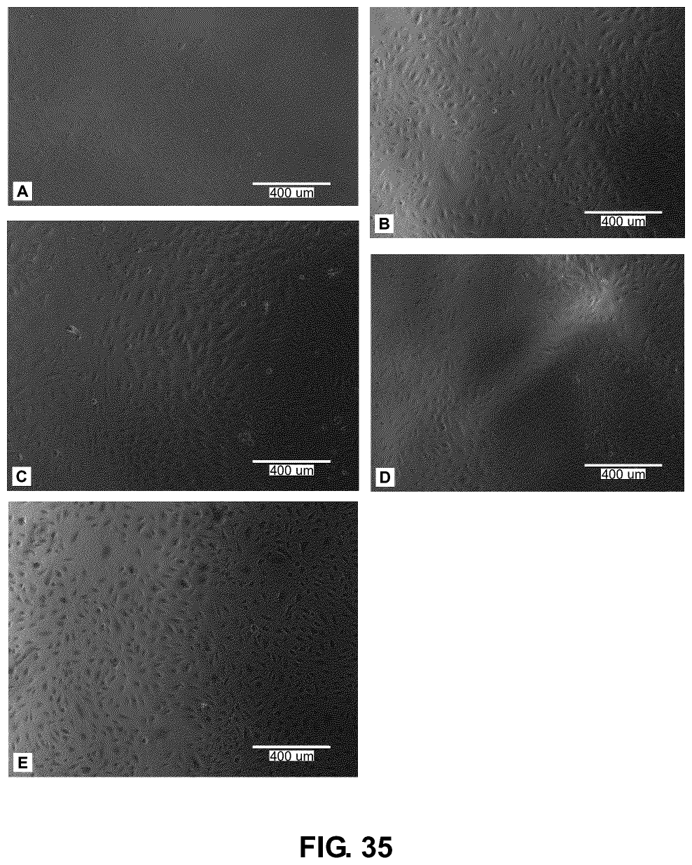

Test wells with test sample identified as “Conventional” (A) before test sample was added, then after introducing insert with test sample at (B) 24 hrs, (C) 48 hrs. (D) 72 hrs, and (E) 1% CV stain. All three test wells of the Conventional sample were similar in appearance and representative images from one well are presented on FIG. 35.

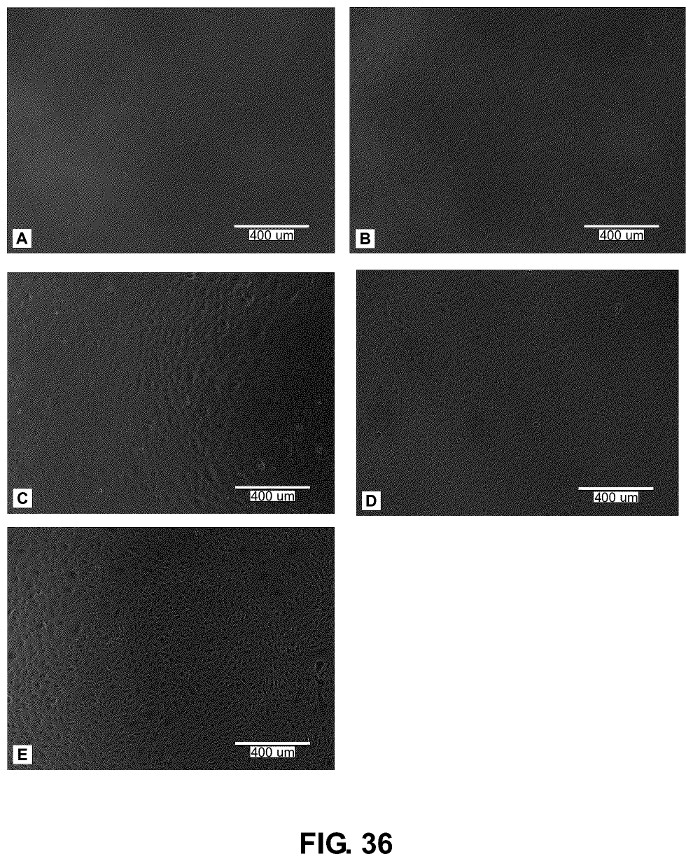

Test wells with test sample identified as an embodiment of the invention (A) before test sample was added, then after introducing insert with test sample at (B) 24 hrs., (C) 48 hrs., (D) 72 hrs., and (E) 1% CV stain. All three test wells of one embodiment of the invention were similar in appearance and representative images from one well are presented in FIG. 36.

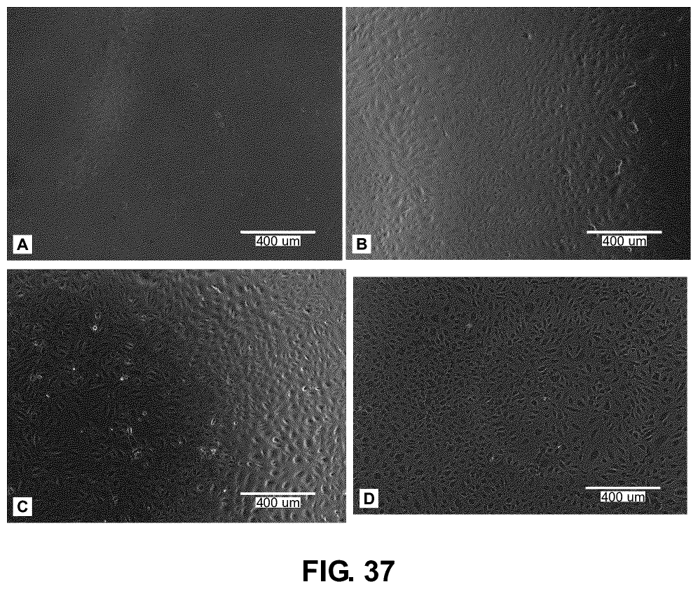

Control wells with primary HDBEC and Complete Growth Medium identified as “Positive Control” A) before test samples were introduced to test wells, then after introducing inserts with test samples to test wells at B) 24 hrs., C) 48 hrs., D) 1% CV stain at 72 hrs. All three control wells were similar in appearance and representative images from one well are presented on FIG. 37.

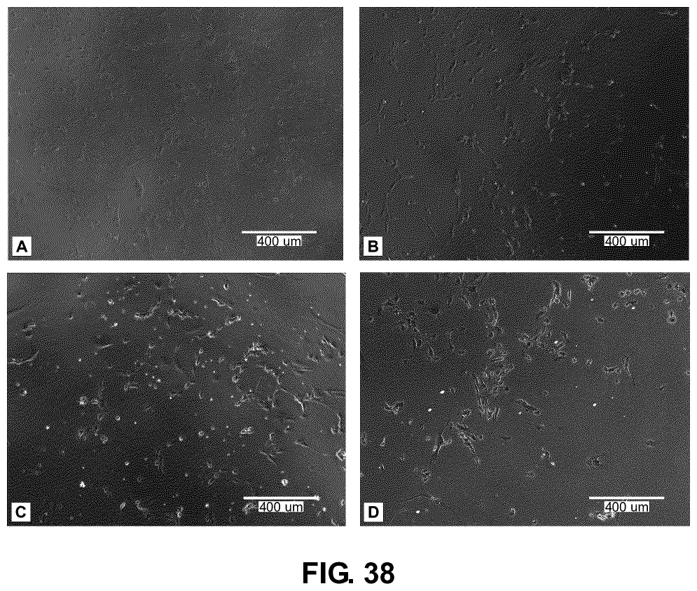

Control wells with primary HDBEC and Basal Media identified as “Negative Control” A) before test samples were introduced to test wells, then after introducing inserts with test samples to test wells at B) 24 hrs., C) 48 hrs., and D) 1% CV stain at 72 hrs. All three control wells were similar in appearance and representative images from one well are presented in FIG. 38.

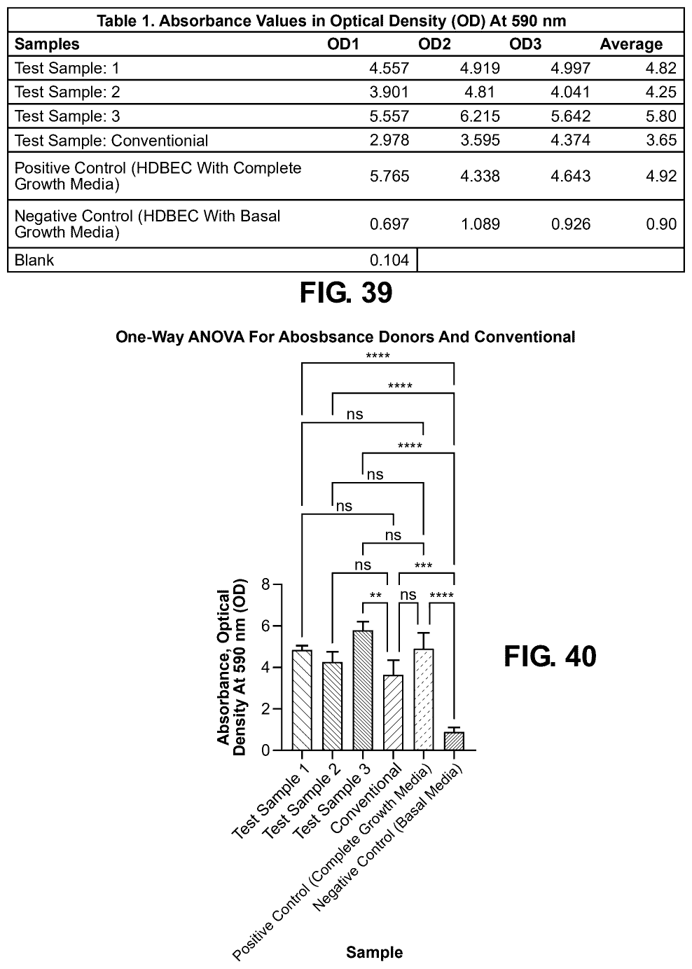

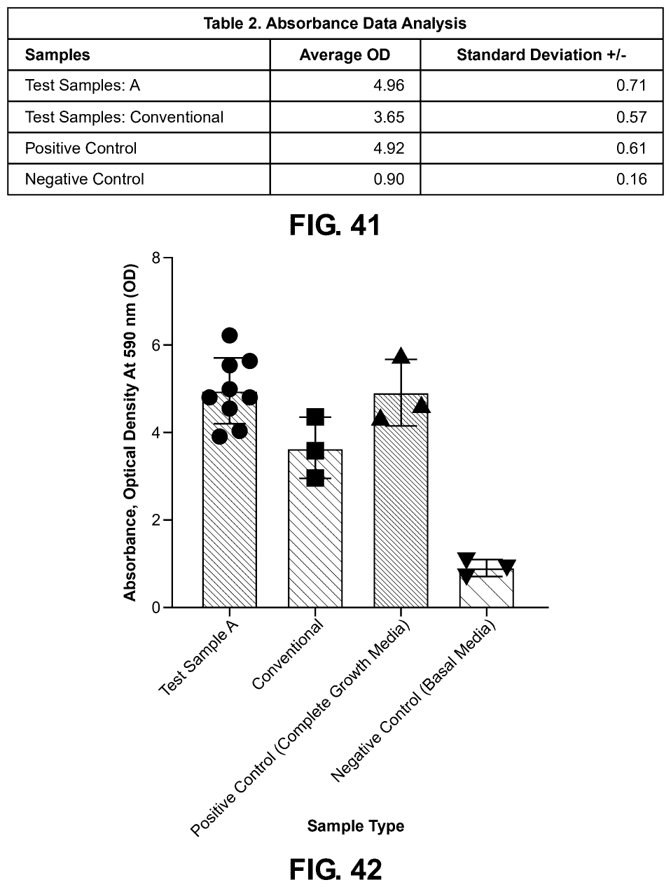

Referring now to FIG. 39-42, the results are now described. Data presented in tables show the absorbance values read from the microplate reader (CLARIOstar). After 3 days in culture, cells were stained with CV for changes in cell number. Levels of Optical Density (OD) show the amount of CV dye eluted from fixed cells (Absorbance—590 nm). No significant differences were observed in total cell number between primary HDBED grown in the presence of a test sample Adipose Allograft, mean OD 4.96±0.71 SD and primary HDBEC grown in full media without test samples, mean OD 4.92±0.61 SD. These results suggest that the presence of test sample Adipose Allograft does not cause a negative effect in the cellular proliferation of primary HDBEC in-vitro. No significant differences in cellular proliferation were observed between the conventional test sample and Adipose Allograft test samples. The test samples, both Adipose Allograft and conventional, did not have a negative effect in the cellular proliferation of primary HDBEC in-vitro. No significant differences in cellular proliferation were observed between the conventional test sample and Adipose Allograft test samples. A significant difference in cellular proliferation of primary HDBEC was observed as expected in positive control, mean OD 4.92±0.61 SD, as compared to negative control, mean OD 0.90±0.16 SD. Cellular proliferation of primary HDBEC was observed in positive control. Cellular proliferation of primary HDBEC was not observed in negative control.

### Experimental Example 6

The purpose of this experiment is to quantify percent adipocyte composition for embodiments of processed tissue described herein.

Human cells, tissues, and cellular and tissue-based product (HCT/P) are defined in Title 21 of the Code of Federal Regulations (CFR) Part 1271 as products containing or consisting of human cells or tissues that are intended for implantation, transplantation, infusion, or transfer into a human recipient (FDA 2020). An HCT/P is regulated solely under section 361 of the PHS act if all set forth criteria are met. One of the criteria for HCT/P regulation is that tissue processing must maintain minimal manipulation.

Minimal manipulation is defined in 21 CFR 1271.10(a) as a processing that does not alter the original relevant characteristics of the tissue relating to the tissue's utility for reconstruction, repair, or replacement. Since adipose tissue is predominantly composed of adipocytes and surrounding connective tissue. The FDA classifies the original relevant characteristics as primarily providing cushioning and support to the body. Therefore, to evaluate whether fabrication of adipose-derived HCT/Ps meets minimal manipulation criteria, it should be considered if the processing alters the HCT/P's utility of providing cushioning and structural support (FDA 2020).

For example, fabrication of an adipose-derived HCT/P where the cellular components are completely removed from the surrounding connective tissue (decellularization) would be considered more than minimally manipulated by the FDA. This is because the decellularization process alters its ability to provide cushioning and support. To show that processed tissue according to embodiments described herein keep with minimal manipulation criteria, this report illustrates how adipocyte structure and connective tissue content are retained.

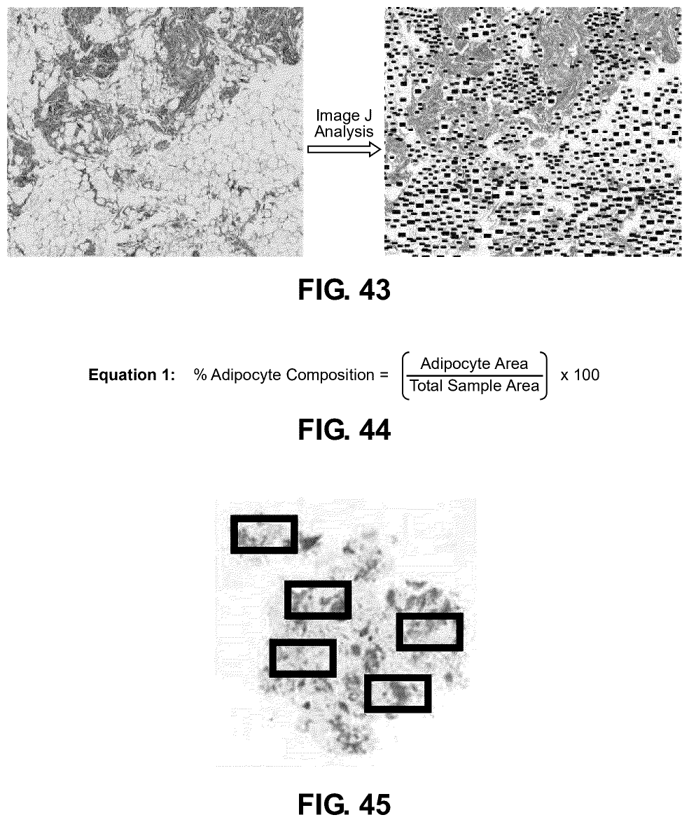

One of the most distinguishable characteristics of the processed tissue is the retention of structurally intact adipocytes. Quantifying adipocyte composition for each product was performed to support the argument of maintaining minimal manipulation. In adipose tissue, hematoxylin and eosin (H&E) staining is a useful tool for distinguishing surrounding connective tissue from adipocytes. Quantitative assessment for processed tissue embodiments were accomplished using the adiposoft plugin for the Fiji adaptation of Image J analysis software (FIG. 43). The adiposoft plugin was designed to identify individual adipocytes and calculate the area inside of each; at which time the software generates a report. By taking the ratio of adipocyte area and total sample area, percent adipocyte composition for each sample can be calculated (Equation 1, FIG. 44).

Adipocyte Structural Quantification: The primary objective of this experiment is to quantify percent adipocyte composition content in adipose allograft samples. This was accomplished through histology staining and Image J analysis.

### Terminology

HCT/P (Human Cell and Tissue Product)
H&E (Hematoxylin and Eosin)

### Materials (Including but not Limited to)

Formalin
5 ml Conical Tubes
Nitrile Gloves
Forceps

### Procedure

(1) Five tissues were fabricated from 5 tissue donors to account for donor variability.
(2) After fabrication, samples were taken from each product iteration, fixed in formalin in 5 mL conical tubes, and submitted for H&E histology staining.
(3) For each histology slide, five images were captured for each sample to obtain a universal characterization (FIG. 45).
(4) For each sample stain, all five images were analyzed via Image J software to measure adipocyte area and total sample area. Average percent adipocyte composition was calculated via Equation 1.

### Results

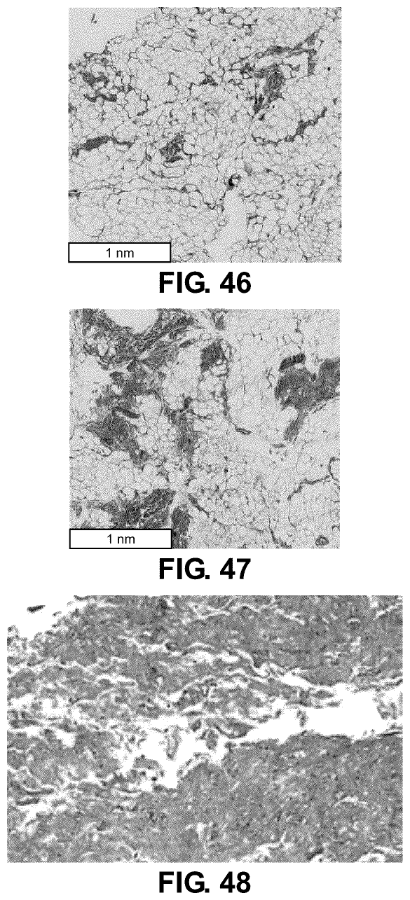

FIGS. 46-48 depict H&E staining images for processed tissues according to first and second processed tissues processed according to embodiments described herein (FIGS. 46 and 47), and a conventional processed tissue (FIG. 48), respectively. For each sample stain, five sections were analyzed via Image J software and the average percent adipocyte composition was calculated.

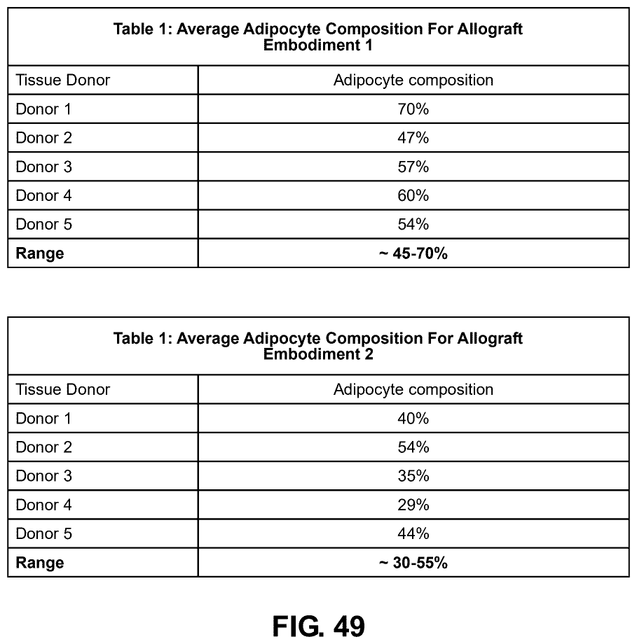

Table 1 (FIG. 49) shows the average results for first tissue samples and table 2 shows average results for second tissue samples. The assessment for each product were sampled from five separate donors. The percent adipocyte range for the first tissue embodiment is calculated to be roughly between 45-70%, while the range for the second tissue embodiment was determined between 30-55%. The difference in adipocyte content may be attributed to the variations in the number of washing steps between the two processing procedures.

### Discussion

Comparison of H&E stains shows both first and second tissue products contain a mixture of extracellular matrix (ECM) components (pink color) and intact adipocytes (pink circles enclosing a white space), while the conventional tissue shows only elements of ECM in their product. This unique feature distinguishes the first and second tissue products from the conventional third product and demonstrates an instrumental advantage in providing cushioning and support, a core characteristic of adipose. Furthermore, this report provides evidence of first/second tissue compliance with HCT/P minimal manipulation requirements through the preservation of adipocyte structure and connective tissue content. There is no processed tissue available that shows retention of both intact adipocytes and connective tissue content. Therefore, both the first and second allograft products incorporate unique structural characteristics not previously developed.

FDA Guidance, Regulatory Considerations for Human Cells, Tissues, and Cellular and Tissue-Based Products: Minimal Manipulation and Homologous Use (July 2020).

Rasband, W.S., ImageJ, U. S. National Institutes of Health, Bethesda, Maryland, USA.

### Experimental Example 7

The purpose of this experiment is to assess adipose tissue composition for processed tissue products according to embodiments described herein. Tissue composition will be evaluated by measuring water content, lipid content, and protein content.

Adipose tissue is composed of connective tissue and various cell populations including adipocytes, pre-adipocytes, endothelial cells, blood cells, fibroblasts, pericytes, and macrophages, and has been increasingly recognized as a major player in metabolic regulation (Luo 2016). The connective tissue is primarily made up of different types of extracellular matrix (ECM) proteins including collagens, glycoproteins, and proteoglycans that are responsible for maintaining structural integrity among other roles. The ECM also controls various physiological processes including cell attachment, differentiation, survival, and development. Furthermore, evidence suggests that the ECM network is required for tissue remodeling and repair (Ruiz-Ojeda 2019).

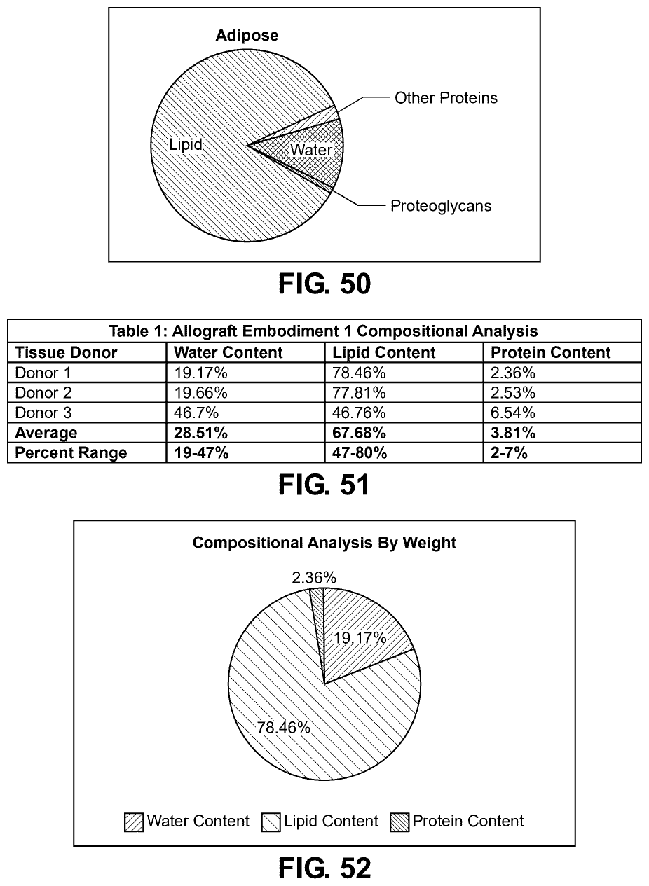

Generally, the three most abundant components of adipose tissue are lipids, ECM proteins, and water. Over the years, many studies have been conducted to measure component quantification in adipose tissue. McKee et al. performed a meta-analysis review of various papers reporting proteomic estimates for absolute and relative amounts of ECM proteins in adipose tissue (McKee 2019). In addition, the authors analyzed empirical data to estimate the amounts of other tissue components including water and lipid content. These amounts were combined with the relative amounts of ECM protein content to determine tissue composition (FIG. 50). According to McKee et al., adipose tissue is typically comprised of 75-80% lipids, 15-20% water, and 3-5% proteins (McKee 2019). The authors' findings of adipose component quantification will be used as a benchmark of expected percentages for all three components in the adipose allograft samples.

### Results

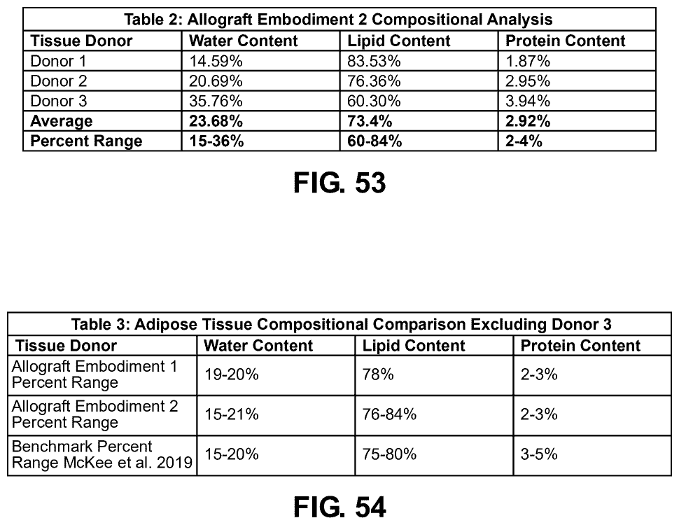

For this experiment, three processed tissues according to one allograft embodiment and three processed tissues according to a second allograft embodiment were fabricated from three tissue donors; with each donor being aliquoted and processed to make both the first and second products. Table 1 (FIG. 51) depicts water, lipid, and protein content percentages for the tested first allograft embodiment samples. The bottom row of Table 1 shows the percent ranges for each component: 19-47% water content, 47-80% lipid content, and 2-7% protein content. FIG. 52 illustrates percent composition for the first product fabricated from tissue donor 1. Table 2 (FIG. 53) shows component quantification for second product samples. The percentages for second product samples are as follows: 15-36% water content, 60-84% lipid content, and 2-4% protein content.

### Discussion of Results

Results show similar component quantifications for first and second tissue samples were similar to published literature concerning adipose tissue, with some slight differences. The first product samples showed a slightly higher water content, but a lower lipid content compared to second product samples. Additionally, the first product samples showed higher protein content range.

A discrepancy in the compositional analysis of donor 3 was noted. Since compositional percentages calculated for this donor did not fall in line with the ranges of the donors 1 and 2, testing was repeated for donor 3 to ensure accuracy of the results which were confirmed to be accurate based on results of the second reading. The data demonstrates donor 3 had a greater amount of connective tissue compared to the other two donors, which may also be responsible for the increased water content in the sample. Furthermore, donor 3 shows a higher protein content compared to donors 1and 2. Based on this information, a compositional range was reported with a single average value to account for the donor variability.
FDA Guidance, Regulatory Considerations for Human Cells, Tissues, and Cellular and Tissue-Based Products: Minimal Manipulation and Homologous Use (July 2020).
McKee T J, Perlman G, Morris M, Komarova S V. Extracellular matrix composition of connective tissues: a systematic review and meta-analysis. Sci Rep. 2019 Jul. 22;9(1):10542. doi: 10.1038/s41598-019-46896-0. PMID: 31332239; PMCID: PMC6646303.
Luo L, Liu M. Adipose tissue in control of metabolism. J Endocrinol. 2016December;231(3):R77-R99. doi: 10.1530/JOE-16-0211. PMID: 27935822; PMCID: PMC7928204.
Ruiz-Ojeda F J, Méndez-Gutiérrez A, Aguilera C M, Plaza-Díaz J. Extracellular Matrix Remodeling of Adipose Tissue in Obesity and Metabolic Diseases. Int J Mol Sci. 2019 Oct. 2;20(19):4888. doi: 10.3390/ijms20194888. PMID: 31581657; PMCID: PMC6801592.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A processed adipose tissue comprising:
an extracellular matrix derived from human adipose tissue; and
a plurality of adipocyte cells having an intact adipocyte composition of at least 10%.

2. The processed adipose tissue of claim 1, wherein the intact adipocyte composition is at least 30%.

3. The processed adipose tissue of claim 1, wherein the intact adipocyte composition is between 30% and 70%.

4. The processed adipose tissue of claim 1, wherein the intact adipocyte composition is between 30% and 55%.

5. The processed adipose tissue of claim 1, wherein the intact adipocyte composition is between 45% and 70%.

6. The processed adipose tissue of claim 1, wherein the extracellular matrix includes a plurality of structural proteins including at least one of collagen, glycoproteins, and proteoglycans retained from the human adipose tissue.

7. The processed adipose tissue of claim 1, wherein the processed adipose tissue is decellularized with a reduced amount of allogeneic components, cellular components, and free lipids.

8. The processed adipose tissue of claim 1 configured for implantation or injection into a patient.

9. The processed adipose tissue of claim 1 configured for passing through an opening between 1 mm and 4.5 mm.

10. A method of processing adipose tissue comprising:
freezing and thawing a human adipose tissue;
separating the human adipose tissue into pieces by mechanical grinding;
serial rinsing the human adipose tissue with a non-ionic surfactant, a crystalloid solution and sterile water; and
dispensing the human adipose tissue into packaging using a loading tool.

11. The method of claim 10, further comprising:
loading the human adipose tissue into a chamber of a container of the loading tool, the chamber having a proximal opening and a distal opening;
actuating a lever to slide a piston distally through the chamber flush along a wall of the chamber to dispense the human adipose tissue into the container, wherein the lever is stabilized with a hinged connection to a stand.

12. The method of claim 11, wherein the proximal opening is at least partially surrounded by a first threaded surface, and the distal opening is at least partially surrounded by a second threaded surface.

13. The method of claim 11, further comprising connecting an adapter having a sealable port to at least one of the proximal opening and the distal opening.

14. The method of claim 11, removing gasses from the human adipose tissue with a sealable port in the piston.

15. The method of claim 10, further comprising debriding the human adipose tissue to remove extraneous or bruised tissue.

16. The method of claim 10, further comprising dispensing the human adipose tissue into centrifuge tubes and centrifuging the human adipose tissue to rinse the human adipose tissue with the non-ionic surfactant.

17. The method of claim 10, further comprising dispensing the human adipose tissue into a sealable container and gently shaking the sealable container to rinse the human adipose tissue with the non-ionic surfactant.

18. The method of claim 10, further comprising rinsing the human adipose tissue in a non-ionic surfactant a second time.

19. The method of claim 10, further comprising rinsing the human adipose tissue in a crystalloid saline solution a second time.

20. The method of claim 10, further comprising rinsing the human adipose tissue in sterile water a second time.

21. The method of claim 10, further comprising drying the human adipose tissue with a drying sieve in a centrifuge tube.

22. The method of claim 10, further comprising decanting the human adipose tissue after each rinse and removing decanted liquid and cellular components.

23. The method of claim 10, wherein the container is a syringe or a cannula.

24. The method of claim 10, further comprising packing the container in a sterile pouch.

25. A media loading tool comprising:
a container having a chamber with a proximal opening and a distal opening;
a piston configured to slide distally and flush along a wall of the chamber;
a lever configured to actuate the piston; and
a stand having a hinged connection to the lever and being configured to stabilize the piston below the lever.

26. The media loading tool of claim 25, wherein the proximal opening is at least partially surrounded by a first threaded surface, and the distal end opening is at least partially surrounded by a second threaded surface.

27. The media loading tool of claim 25, further comprising an adapter having a sealable portion, wherein the adapter is configured to connect to the distal opening of the container.

28. The media loading tool of claim 25, wherein the piston has a sealable port.
